(19) United States
(12) Patent Application Publication
(10) Pub. No.: US 2021/0085699 A1
(43) Pub. Date: Mar. 25, 2021

(54) COMBINATION THERAPIES FOR TREATING CANCERS

(71) Applicant: The Jackson Laboratory, Bar Harbor, ME (US)

(72) Inventors: Muneer G. Hasham, Bar Harbor, ME (US); Derry C. Roopenian, Bar Harbor, ME (US)

(73) Assignee: The Jackson Laboratory, Bar Harbor, ME (US)

(21) Appl. No.: 16/971,887

(22) PCT Filed: Feb. 21, 2019
(86) PCT No.: PCT/US2019/018879

§ 371 Date: Aug. 21, 2020

Related U.S. Application Data
(60) Provisional application No. 62/765,028, filed on Aug. 17, 2018.
(60) Provisional application No. 62/634,400, filed on Feb. 23, 2018.

(51) Int. Cl. A61K 31/7004; A61K 31/275; A61P 35/00; A61P 35/02

(52) U.S. Cl. CPC A61K 31/7004; A61P 35/02; A61P 35/00; A61K 31/275

(57) ABSTRACT

Provided herein are compositions, methods, and kits for anticancer therapies using a glycolytic inhibitor and a RAD51 complex inhibitor.

### RELATED APPLICATIONS

This application claims the benefit under 35 U.S.C. § 119(e) of U.S. provisional application No. 62/765,028, filed Aug. 17, 2018, and U.S. provisional application No. 62/634,400, filed Feb. 23, 2018, each of which is incorporated by reference herein in its entirety.

### BACKGROUND

Glycolysis is a major ATP-producing pathway in mammalian cells, and can lead to either lactate fermentation or pyruvate oxidation, with lactate fermentation yielding significantly fewer ATP molecules per molecule of glucose metabolized (1). Cancer cells are characterized by a high rate of glycolysis as compared to normal cells, leading to excessive lactate fermentation despite inefficient ATP production, a phenomenon termed the Warburg Effect (2-4). This effect was initially thought to be a cause of neoplasticity, but now is considered a key feature of neoplastic cells in numerous types of cancers (5, 6).

Because of the increased use of glycolysis in cancer cells compared to non-neoplastic cells, glycolytic inhibitors have been considered an attractive means of targeting cancer (7). Nonetheless, because glycolysis is a universal metabolic pathway, its blockade by glycolytic inhibitors such as 2-deoxy-glucose (2DG) at doses that are efficacious can yield adverse side effects (8-10). Therefore, glycolytic inhibitors have been tested at low doses in combination with other cytotoxic therapies (reviewed in (10)). While this combination approach has been shown to be successful in targeting a number of malignancies, most compounds used in combination with glycolytic inhibitors are not tumor-specific and can therefore damage non-malignant cells (10). In addition, such compounds can also potentially introduce DNA damage that can lead to mutations or breaks, potentially resulting in neomalignancies (reviewed in (10)).

### SUMMARY

Provided herein, are compositions, methods, and kits for anticancer therapies. The disclosure herein combines two avenues of targeting cancer cells—metabolism and DNA repair—and shows that targeting these two areas in combination produces a synthetic lethal effect that is more effective than the current standard-of-care treatment. Specifically, an approach that effectively targets cancer cells through the inhibition of glycolysis and specific inhibition of RAD51 is described. This combination of inhibitors results in an unexpected synergistic effect on cancer cells (see, e.g., Example 6).

Targeting the early steps of the glycolysis pathway in cancers is a well-established therapeutic strategy; however, the doses required to elicit a therapeutic effect on the cancer can be toxic to the patient. Consequently, numerous preclinical and clinical studies have combined glycolytic blockade with other therapies. However, most of these other therapies do not specifically target cancer cells, and thus they also affect normal tissue. With prolonged treatment, these combined therapies could give rise to adverse effects.

Provided herein are a diverse number of cancer models—spontaneous, patient-derived xenografted tumor samples, or xenografted human cancer cells—that can be efficiently targeted by 2-deoxy-D-Glucose (2DG), a non-specific glycolytic inhibitor. The cancer-cell specificity of a therapeutic compound using the MEC1 cell line, a chronic lymphocytic leukemia (CLL) cell line that expresses activation induced cytidine deaminase (AID) was also tested. Data presented herein has further shown that MEC1 cells were susceptible to 4,4′-Diisothiocyano-2,2′-stilbenedisulfonic acid (DIDS), a specific RAD51 inhibitor. Although RAD51 is required for normal cell function, as it is required for homologous recombination repair, DIDS did not have an adverse effect on AID-negative MEC1 cells, indicating that this RAD51 inhibitor affects only AID-positive cells.

The glycolytic inhibitor, 2DG, was also evlauted to alleviate tumor burden in spontaneous and patient-derived xenograft (PDX) cancer mouse models. Furthermore, data shown here has demonstrated that DIDS reduced tumor burden in xenografted cell lines in vivo. Ultimately, the efficacy of DIDS in reducing tumor burden in vivo in mice was enhanced by the effect of 2DG; both used at dosages that lowerd the risk of adverse effects, indicating that the combination of RAD51 inhibition and glycolytic blockage is an effective therapy for AID-positive cancers.

When 2DG and DIDS were combined, each at a lower dose than that used when administered alone, this combination was more efficacious than fludarabine, the current standard-of care treatment for CLL. This suggested that the blockade of glycolysis by 2DG, together with the inhibition of homologous recombination by RAD51 with compounds such as DIDS, can be a potentially beneficial combination for targeting AID positive cancer cells with minimal adverse effects on normal tissue.

The glycolytic inhibitor 2DG, and RAD51 inhibitor DIDS act synergistically to reduce B-lymphocyte human chronic lymphocytic leukemia proliferation ex vivo. In addition, mice xenografted with this cell line showed significant reduction in the tumor burden in the presence of both 2DG and DIDS. These data suggest that the glycolytic pathway and DNA repair can be simultaneously targeted to produce better chemotherapies against B-lymphoid cancers.

Thus, some aspects of the present disclosure comprise methods comprising administering to a subject (e.g., a human subject) a glycolytic inhibitor and a RAD51 complex inhibitor. In some embodiments, the glycolytic inhibitor is 2-deoxy-D-Glucose (2DG). In some embodiments, the RAD51 complex inhibitor is selected from 4,4′-Diisothiocyano-2,2′-stilbenedisulfonic acid (DIDS), B02, and RI-1. In some embodiments, the glycolytic inhibitor is 2DG and the RAD51 complex inhibitor is DIDS.

The subject, in some embodiments, has a cancer. In some embodiments, the cancer is a glycolysis-dependent cancer. In some embodiments, the cancer expresses activation induced cytidine deaminase (AID). In some embodiments, the cancer is a B-cell cancer. For example, the B-cell cancer may be a leukemia. In some embodiments, the leukemia is chronic lymphocytic leukemia (CLL).

Other aspects of the present disclosure provide methods comprising contacting a cell with a glycolytic inhibitor and a RAD51 complex inhibitor. In some embodiments, the glycolytic inhibitor is 2DG and the RAD51 complex inhibitor is DIDS.

The cell, in some embodiments, is a cancer cell. For example, the cancer cell may be a glycolysis-dependent cancer cell. In some embodiments, the cancer cell expresses AID. In some embodiments, the cancer cell is a B-cell cancer cell. For example, the B-cell cancer cell may be a leukemia cell. In some embodiments, the leukemia cell is a CLL cell.

Also provided herein are compositions comprising a glycolytic inhibitor and a RAD51 complex inhibitor. In some embodiments, the glycolytic inhibitor is 2DG and the RAD51 complex inhibitor is DIDS. In some embodiments, the glycolytic inhibitor and the RAD51 complex inhibitor are formulated in the composition at a dose that is lower than a control standard-of-care dose.

In some embodiments, the glycolytic inhibitor and the RAD51 complex inhibitor are administered sequentially. In other embodiments, the glycolytic inhibitor and the RAD51 complex inhibitor are administered concurrently.

In some embodiments, the glycolytic inhibitor and the RAD51 complex inhibitor are administered in an amount effective for reducing the number of CD19⁺ cells in the subject, relative to a control. In some embodiments, the glycolytic inhibitor and the RAD51 complex inhibitor are administered at doses that are lower than a control standard-of-care dose. In some embodiments, the control standard-of-care dose is a standard-of-care dose of fludarabine.

Also provided herein are kits comprising a glycolytic inhibitor, a RAD51 complex inhibitor, and optionally one or more delivery device (e.g., needle/syringe).

### BRIEF DESCRIPTION OF THE DRAWINGS

FIGS. 1A-FIG. 1E provide data indicating that 2-deoxy-D-Glucose (2DG) alleviated tumor burden in a spontaneous mouse cancer model. FIG. 1A are representative images showing inguinal lymph node tumor reduction on an SJL mouse treated with 2DG dissolved in drinking water (6 g/L); photos of a mouse with a greatly enlarged spleen shrunk with 2DG treatment by 1 week, and returned to normal appearance after two weeks of treatment are depicted. FIG. 1B are pie charts showing the transient effect of 2DG on tumor regression, and subsequent re-emergence (N=7). While 6 of the 7 mice responded well to the 2DG treatment, approximately ¾ of the mice saw tumor resurgence after 5-11 weeks of treatment (N=7). FIG. 1C are Computed tomography (CT) scans of a p53 wildtype (Trp53+/+) and p53 mutant (Trp53−/−) mouse, showing the maximum engulfment of a thymic lymphoma in the chest cavity. FIG. 1D is a survival curve of Trp53−/− mice treated with 2DG (670 mg/kg) or glucose (control) three times per week via intraperitoneal injections. FIG. 1E is a graph showing weights of mice during glucose or 2DG treatment.

FIGS. 2A and 2B provide data showing that glycolysis sensitivity rather than proliferation determined susceptibility to 2DG in lung PDX tumor models. FIG. 2A is a bar graph showing that glycolysis-dependent (TM00921) and glycolysis-independent (TM00244) PDX lung tumors were subcutaneously xenografted in NSG™ mice, and tumor growth was measured for 4 weeks. Growth was normalized to the initial size of the tumor for each individual mouse (N=4-5) and plotted as fold increase. FIG. 2B are graphs showing the growth of the two lung tumors in mice treated with either 2DG or glucose (control).

FIGS. 3A-3D provide data showing that the glycolysis-dependent MEC1 cell line was susceptible to 2DG in vivo. FIG. 3A is a graph of the metabolic comparison of two AID-positive B-cell lines, MEC1 and CCRF-SB. CCRF—SB and MEC1 showed equal respiration but different levels of glycolysis. NRG mice were xenografted with either CCRF-SB or MEC1 cells for two weeks, treated with 600 mg/kg of 2DG for two weeks and then measured for burden by flow cytometry. FIG. 3B is a graph showing that the two AID-positive B-cell lines, MEC1 and CCRF-SB had similar growth rates ex vivo. N=3 in two experiments. FIG. 3C are bar graphs showing bone marrow and spleen tumor burden as measured by human CD19+ cells treated with glucose or 2DG. N=4 mice. FIG. 3D is a graph of a comparison of free glucose inside spleen and bone marrow cells of mice treated with 2DG or Glucose (6 g/L) or regular water (vehicle) for 1 week (N=5 Mice per group).

FIGS. 4A-4F provide data showing that the RAD51-sensitive MEC1 cell line was targeted by the RAD51 inhibitor DIDS in vivo. FIG. 4A is a bar graph of quantitative RT-PCR of AICDA (AID) and RAD51 of MEC1, K562 (negative control for AID), and CCRF-SB cells. FIG. 4B is flow cytometry of intracellular staining of AID in the aforementioned cell lines (K562, MEC1 and CCRF-SB cells). FIG. 4C is a graph of the ex vivo growth of MEC1 B-cells in the presence of different doses of DIDS. FIG. 4D is a graph of MEC1 tumor burden in NRG™ mice after two-week treatment with DIDS as measured by hCD19+ cells in the spleen. Each point represents a mouse (data pooled from 3 experiments). FIG. 4E is a bar graph showing tumor burden of MEC1 cells and MEC1 cells without AID (AKO) after DIDS treatment (N=5 to 9 mice). FIG. 4F are bar graphs of in vivo treatment of AID−/− mice with different concentrations of DIDS, followed by treatment of splenocytes with radiation ex vivo, illustrating the capacity of DIDS to work in combination treatment with IR (N=5 mice).

FIGS. 5A-5E provide data showing that the glycolysis blockade and RAD51 inhibition exhibited a synergistic and enhanced anti-cancer effect on tumor burden. Both DIDS and 2DG reduce MEC1 cells in vivo but at high doses of each of the compounds. Together, at lower doses, DIDS and 2DG showed a synergistic effect on MEC1 cells ex vivo and in vivo. FIG. 5A is a graph showing the ex vivo survival of MEC1 cells incubated with different doses of DIDS and 2DG 48 hours after incubation. FIG. 5B is a heat map of the proliferation of MEC1 and SuDHL cells treated with 2DG or DIDS ex vivo. The color shows the numbers (in millions) of cells after 5 days of proliferation. Initial number for MEC1 was 1 million cells and SuDHL was 0.5 million cells. FIG. 5C are bar graphs of in vivo MEC1 tumor burden as measured by the percentage of hCD19+ cells in the spleen and bone marrow after two-week treatment with low doses of DIDS and/or 2DG, with fludarabine, or with glucose (N=4 to 13 mice in two experiments). FIG. 5D is a bar graph showing the average weight of the mice in each group at the end of the treatment regimen. FIG. 5E is a bar graph showing ex vivo survival of MEC1 cells incubated in 2DG at varying concentrations for 24 hours (N=3).

FIG. 6 are images showing a colorized 3D rendering of micro-computed tomography images of control Trp53+/+ and a Trp53−/− mouse with a thymic lymphoma tumor.

FIGS. 7A-7D are data of the generation and validation of AID knock-out (AKO) MEC1 cells. FIG. 7A is a schematic of the knockout. FIG. 7B is an image of a gel of the PCR validation of AKO. FIG. 7C are graphs showing the sensitivity of AKO cells and parental MEC1 cells (AID+ cells) to DIDS. FIG. 7D is a graph showing the spleen weights of MEC1 cell-engrafted and AKO cell-engrafted mice treated with DIDS.

FIG. 8 shows titration curves of MEC1 and SuDHL cells with DIDS and B02 RAD51 inhibitors. The top left graph shows a titration curve of MEC1 cells with DIDS. The top right graph shows a titration curve of MEC1 cells with B02. The bottom left graph shows a titration curve of SuDHL cells with DIDS. The bottom right graph shows a titration curve of SuDHL cells with B02.

FIG. 9 is a schematic of the model described herein.

FIG. 10 are data showing that a high dose of DIDS is required to reduce tumor burden in vivo. NRG mice were xenografted with MEC1 or MEC1 LUC for two weeks, and then treated at 50 mg/kg DIDS or with vehicle for two weeks. Mice were imaged for the expression of the luciferase gene as a measurement of tumor burden. The graph showed tumor burden as measured by flow cytometry in the bone marrow and spleen of mice treated with vehicle, DIDS or fludarabine.

### DETAILED DESCRIPTION

The present disclosure provides, in some embodiments, methods, compositions, and kits for treating cancer by specifically inhibiting glycolysis and RAD51. In some embodiments, the methods comprise contacting a cancer cell with (e.g., exposing a cancer cell to) a glycolytic inhibitor and a RAD51 complex inhibitor. In other embodiments, the methods comprise administering to a subject having (e.g., diagnosed with) cancer a glycolytic inhibitor and a RAD51 complex inhibitor. The data provided herein shows that targeting both metabolism and DNA repair specifically in cancer cells is more effective than the non-specific standard-of-care treatments, which often results in adverse side effects associated with toxicity.

### Glycolytic Inhibition

Cells use many pathways to produce energy. Under aerobic conditions, cells preferentially use oxygen to produce energy in the form of adenosine triphosphate (ATP) in a process known as oxidative phosphorylation. Under anaerobic conditions, cells can perform glycolysis to produce ATP without oxygen. Glycolysis is a sequence of reactions that converts glucose into pyruvate, which is ultimately converted into energy in the form of ATP. While cancer cells are growing and the tumor is expanding, there is reduced oxygen present due to limited vascularization in the tumor. Non-cancer cells preferentially use oxygen to produce ATP. Thus, without being bound by theory, inhibiting glycolysis may preferentially prevent the growth and spread of cancer cells and tumors, without affecting non-cancer cells.

A glycolytic inhibitor is an agent (e.g., nucleic acid, protein, chemical) that decreases the glycolytic activity in a cell. Cells with high metabolic activity, such as cancer cells, perform more glycolysis to produce increased energy compared to cells with low metabolic activity. Glycolytic inhibitors can target different steps of glycolysis. In some embodiments, a glycolytic inhibitor blocks the activity of enzymes. In some embodiments, a glycolytic inhibitor is a substrate for glycolysis that is not able to metabolized into pyruvate, resulting in futile cycling. In some embodiments, a glycolytic inhibitor blocks the formation of necessary substrates in glycolysis. Non-limiting examples of glycolytic inhibitors of the present disclosure include 2-deoxy-D-glucose (2DG) (see, e.g., Pelicano H. et al. Oncogene 2006; 25(34): 4633-4646), Phloretin, Quercetin, STF31, WZB117, 3-3-pyridinyl)-1-(4-pyridinyl)-2-progen-1-one (3PO), 3-bromopyruvate, Dichloroacetate, Oxamic acid, NHI-1, 6-aminonicotinamide, lonidamine, oxythiamine chloride hydrochloride, and shikonin.

In some embodiments, the glycolytic inhibitor is 2DG. 2DG is a synthetic glucose analog molecule in which the 2-hydroxyl group is replaced by hydrogen, which prevents its further metabolism in glycolysis. Therefore, 2DG inhibits glycolysis by triggering futile cycling. Futile cycling refers to inhibiting a pathway (e.g., glycolysis) using of a substrate analog that is bound by an enzyme but cannot be metabolized. 2DG is taken up by cells by glucose transporters. Cells with higher glucose uptake (e.g., cancer cells), will also have a higher uptake of 2DG.

### RAD51 Complex Inhibition

Cancer cells grow and divide more rapidly than non-cancer cells. Cell growth and division is inhibited when genomic DNA is damaged and not repaired. Numerous DNA damage repair proteins, including RAD51, BRCA1, and BRCA2, are overexpressed in some cancer cells. Thus, without being bound by theory, inhibiting DNA damage repair may preferentially inhibit the cell growth and division of cancer cells compared to non-cancer cells.

There are numerous DNA damage repair pathways in eukaryotes (e.g., homologous recombination, interstrand cross-link repair) that require the RAD51 protein and/or the RAD51 complex of proteins to repair DNA double strand breaks (DSBs). RAD51 is a eukaryotic protein encoded by the RAD51 gene which is highly similar to the bacterial RecA and Saccharomyces cerevisiae Rad51 proteins. RAD51 polymers form a nucleoprotein filament on damaged ssDNA, protecting it from degradation and recruiting other DNA damage repair proteins to form RAD51 protein complexes. RAD51 is overexpressed in some cancer cells, where it confers resistance to treatment by promoting DNA damage repair.

RAD51 protein complexes comprise RAD51 and at least one other protein and are required for the repair of DNA DSBs. Non-limiting examples of Rad51 complexes include Rad51, replication protein A (RPA), and Rad52; Rad51, PALB2, and RAD51C; RAD51C and XRCC3; Rad51 and BRCA1; Rad51 and BRCA2; Rad51, XRCC3, and Rad54; PALB2, BRCA2, RAD51C, and XRCC3.

A RAD51 complex inhibitor is an agent (e.g., nucleic acid, protein, chemical) that decreases the activity of a RAD51 protein complex. In some embodiments, RAD51 complex inhibitor decreases the formation of a RAD51 polymer. In some embodiments, RAD51 complex inhibitor decreases ATP hydrolysis by RAD51. In some embodiments, RAD51 complex inhibitor decreases RAD51 binding single-stranded DNA (ssDNA). Non-limiting examples of RAD51 inhibitors of the present disclosure include 4,4′-Diisothiocyano-2,2′-stilbenedisulfonic acid (DIDS), (E)-3-benzyl-2-(2-(pyridine-3-yl) vinyl) quinazolin-4(3H)-one (B02) (see, e.g., Huang F. et al. PLOS ONE, 2014; 9(6): e100933), 3-chloro-1-(3,4-dichlorophenyl)-4-(4-morpholinyl)-1H-pyrrole-2,5-dione (RI-1) (see, e.g., Bedke B. et al. Nucleic Acids Res. 2012; 40(15): 7347-57), IBR2 (see, e.g., Ferguson P. J. et al. J Pharmacol Exp Ther. 2018; 364(1): 46-54), IBR121 and IBR120 (see, e.g., Zhu J. et al. Eur J Med Chem. 2015; 96: 196-208). See also WO2016140971A1, published Sep. 9, 2016).

In some embodiments, the RAD51 inhibitor is DIDS, which inhibits Rad51-mediated strand invasion as well as RAD51-mediated pairing of homologous sequences in the absence of RPA. DIDS binds directly to RAD51 and significantly inhibits RAD51 binding to DNA. Thus, DIDS may bind near the DNA binding site of RAD51 and compete with DNA for binding RAD51.

In some embodiments, the RAD51 inhibitor is B02, which efficiently and specifically decreases the DNA strand exchange activity of RAD51. Specifically, B02 impairs the RAD51-single stranded DNA interaction at the primary site of RAD51 during nucleoprotein filament formation and its secondary DNA binding site, where dsDNA binds during the search for a homologous DNA sequence.

In some embodiments, the RAD51 inhibitor is 3RI-1, which possess a chloromaleimide moiety which covalently binds to the thiol group in cysteine-319 of RAD51, thus blocking the interface between monomeric RAD51 proteins and an ATP-binding loop. Thus, RI-1 alters RAD51-ATP interactions and inhibits RAD51-RAD51 monomer binding and polymerization, which is essential for filament formation and elongation.

In some embodiments, the RAD51 inhibitor is selected from DIDS, B02, and RI-1.

### Cancer/Cancer Cells

The present disclosure provides methods of contacting a cell, such as a cancer cell, with a glycolytic inhibitor and a RAD51 complex inhibitor. In some embodiments, the cancer exhibits increased glycolysis (e.g., neurons, cancer cells) and/or increased DNA damage repair (e.g., stem cells, cancer cells) compared to a control cell (e.g., a non-cancer cell). In some embodiments, the cancer cell is resistant to treatment with only a glycolytic inhibitor. In some embodiments, the cancer cell is resistant to treatment with only a RAD51 complex inhibitor. A cell is considered to be resistant to an agent, such as a glycolytic inhibitor or a RAD51 complex inhibitor, if it survives in the presence of the agent.

Non-limiting examples of cancers/cancer cells that may be treated/contacted with glycolytic inhibitors and RAD51 complex inhibitors include: leukemia cells, such chronic lymphocytic leukemia (CLL) and chronic myeloid leukemia (CML); lymphoma cells, such as Hodgkin and non-Hodgkin lymphoma; breast cancer cells, pancreatic cancer cells, lung cancer cells, melanoma cells, colorectal cancer cells, stomach cancer cells, renal cancer cells, brain cancer cells, liver cancer cells, bladder cancer cells, prostate cancer cells, uterine cancer cells, ovarian cancer cells, cervical cancer cells, testicular cancer cells, head and neck cancer cells, multiple myeloma cells, thyroid cancer cells, and carcinoid cancer cells. Other cancer cells are contemplated herein.

In some embodiments, a cancer cell is considered to be a glycolysis-dependent cell. Cancer cells consume glucose, perform glycolysis, and generate ATP at a much faster rate than non-cancer cells, even in aerobic environments. In aerobic conditions, non-cancer cells preferentially produce ATP through oxidative phosphorylation instead of glycolysis. Increased glucose consumption, particularly under aerobic conditions, can be used to detect cancer cells, and targeting this difference in metabolism between cancer and non-cancer cells may be exploited as described herein.

In some embodiments, the cancer cell expresses activation induced cytidine (AID). AID is an enzyme in humans which is encoded by the AICDA gene and generates mutations in DNA through deamination of a cytosine (C) base to a uracil (U) (which is recognized as a thymine (T). Thus, after cytosine deamination, a C:G base pair is mutated into a U:G mismatch. Because a U is recognized as a T by DNA polymerase proteins, a U:G mismatch is subsequently converted to a T:A base pair as a result of cytosine deamination by AID. In the B cells of lymph nodes, AID causes mutations which produce antibody diversity, but can also produce mutations, which lead to B cell cancers.

In some embodiments, a cancer is a B cell cancer. A B cell cancer affects the B cells that originate in the lymph nodes before moving into the blood stream. In some embodiments, a B cell cancer is leukemia. Leukemia is a cancer that starts in the blood-forming cells of the bone marrow, when cells no longer mature properly and proliferate rapidly. Leukemia cells build up in the bone marrow, crowding out the healthy cells. Lymphocytic leukemia begins in lymphocyte precursors. Unlike in lymphomas, in which the cancer cells are mainly in the lymph nodes and other tissues, in lymphocytic leukemia the cancer cells are mainly in the bone marrow and the blood.

In some embodiments, the leukemia is chronic lymphocytic leukemia (CLL). CLL is a type of cancer which begins with cells in the bone marrow. CLL results in the build-up of B cell lymphocytes in the bone marrow, lymph nodes, and blood. These cells do not function well and crowd out the healthy blood cells. CLL is divided into two main types: those with a mutated immunoglobulin variable-region heavy chain (IGHV) gene and those without a mutated IGHV gene. High-risk patients have a pattern of immature B cells with few mutations in the DNA in the IGHV antibody gene region, whereas low-risk patients show considerable mutations of the DNA in the antibody gene region indicating mature lymphocytes.

### Compositions and Treatment Methods

Some aspects of the disclosure provide methods for treating cancer with a glycolytic inhibitor and a RAD51 complex inhibitor. In some embodiments, a method comprises administering to a subject (e.g., a human subject having cancer) a glycolytic inhibitor (e.g., 2DG) and a RAD51 complex inhibitor (e.g., DIDS, B02, or RI-1). The glycolytic inhibitor and the RAD51 complex inhibitor may be administered by any route known in the art. Non-limiting examples of routes of administration include: oral (e.g., tablet, capsule), intravenous, subcutaneous, inhalation, intranasal, intrathecal, intramuscular, intraarterial, and intraneural.

Compositions of the present disclosure (e.g., for use in a method of treatment) may comprise a glycolytic inhibitor (e.g., 2DG) and a RAD51 complex inhibitor (e.g., (DIDS), B02, or RI-1). Thus, in some embodiments, the glycolytic inhibitor and the RAD51 complex inhibitor are co-formulated (present in the same composition). In some embodiments, a composition is administered in an effective amount.

An effective amount, which may also be referred to as a therapeutically effective amount, refers to the amount (e.g., dose) at which a desired clinical result (e.g., cancer cell death) is achieved in a subject. An effective amount is based, at least in part, on the target tissue, target cell type, means of administration, physical characteristics of the inhibitor, other components of the composition, and other determinants, such as age, body weight, height, sex and general health of the subject. A subject may be a mammal, such as a human, a non-human primate (e.g., Rhesus monkey, chimpanzee), or a rodent (e.g., a mouse or a rat), In some embodiments, the subject is a human subject. In some embodiments, the subject has a cancer. In some embodiments, the subject has a glycolysis-dependent cancer. In some embodiments, the subject has a cancer that expresses AID.

In some embodiments, a composition is a pharmaceutical composition. A pharmaceutical composition is a combination of an active agent, such as a glycolytic inhibitor and/or RAD51 complex inhibitor with a carrier, inert or active, making the composition especially suitable for therapeutic use in vivo or ex vivo. A pharmaceutically acceptable carrier after administered to or upon a subject, does not cause undesirable physiological effects. The carrier in the pharmaceutical composition must be acceptable also in the sense that it is compatible with the active agent and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Formulations described herein may be prepared by any method known or hereafter developed in the art of pharmacology. In general, such preparatory methods include the step of bringing the active ingredient (e.g., inhibitor) into association with an excipient and/or one or more other accessory ingredients, and then, if necessary and/or desirable, dividing, shaping and/or packaging the product into a desired single-dose or multi-dose unit.

Relative amounts of the active agent, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient.

The ratio of glycolytic inhibitor to RAD51 complex inhibitor in an composition may vary. In some embodiments, the ratio of glycolytic inhibitor to RAD51 complex inhibitor is 1:1 to 1:10, or 1:1 to 1:5. For example, the ratio of glycolytic inhibitor to RAD51 complex inhibitor may be 1:1, 1:2, 1:3, 1:4, 1:5, 1:6, 1:7, 1:8, 1:9, or 1:10. In other embodiments, the ratio of RAD51 complex inhibitor to glycolytic inhibitor is 1:1 to 1:10, or 1:1 to 1:5. For example, the ratio of RAD51 complex inhibitor to glycolytic inhibitor may be 1:1, 1:2, 1:3, 1:4, 1:5, 1:6, 1:7, 1:8, 1:9, or 1:10.

In some embodiments, the glycolytic inhibitor and the RAD51 complex inhibitor are administered sequentially. For example, the glycolytic inhibitor may be administered before or after (e.g., on the order of minutes, hours, or days before or after) the RAD51 complex inhibitor.

In some embodiments, the glycolytic inhibitor and the RAD51 complex inhibitor are administered concomitantly (at the same time). For example, the glycolytic inhibitor and the RAD51 complex inhibitor may be formulated in the same composition.

In some embodiments, the dose of glycolytic inhibitor and/or RAD51 complex inhibitor administered to a subject is equivalent to or lower than a control standard-of-care dose. In some embodiments, the dose of the glycolytic inhibitor is lower than a control standard-of-care dose. In some embodiments, the dose of the RAD51 complex inhibitor is lower than a control standard-of-care dose. A standard of care refers to a medical treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A standard-of-care dose as provided herein refers to the dose of glycolytic inhibitor and/or RAD51 complex inhibitor that a physician/clinician or other medical professional would administer to a subject to treat or prevent cancer, while following the standard of care guideline for treating or preventing cancer.

In some embodiments, the dose of glycolytic inhibitor administered to a subject is a standard-of-care dose. In some embodiments, the dose of glycolytic inhibitor administered to a subject is at least 10% less than the standard-of-care dose for the glycolytic inhibitor. For example, the dose of glycolytic inhibitor administered to a subject is at least 15%, at least 20%, at least 30%, at least 40%, or at least 50% less than the standard-of-care dose for the glycolytic inhibitor. In some embodiments, the dose of glycolytic inhibitor administered to a subject is 10%-50%, 10%-40%, 10%-30%, 10%-20%, 20%-50%, 20%-40%, 20%-30%, 30%-50%, 30%-40%, or 40%-50% less than the standard-of-care dose for the glycolytic inhibitor.

In some embodiments, the glycolytic inhibitor 2DG is administered to a subject at a dose of 30 mg/kg (ClinicalTrials.gov No.: NCT00633087), or at a dose of less than 30 mg/kg. In some embodiments, the glycolytic inhibitor 2DG is administered to a subject at a dose of 45 mg/kg, or at a dose of less than 45 mg/kg. In some embodiments, the glycolytic inhibitor 2DG is administered to a subject at a dose of 60 mg/kg, or at a dose of less than 60 mg/kg. In some embodiments, the glycolytic inhibitor 2DG is administered to a subject at a dose of 10-100 mg/kg. In some embodiments, the glycolytic inhibitor 2DG is administered to a subject at a dose of 10, 15, 20, 25, 30, 35, 40, 45, 50, 55, 60, 65, 70, 75, 80, 85, 90, 95, or 100 mg/kg.

In some embodiments, the dose of RAD51 complex inhibitor administered to a subject is a standard-of-care dose. In some embodiments, the dose of RAD51 complex inhibitor administered to a subject is at least 10% less than the standard-of-care dose for the RAD51 complex inhibitor. For example, the dose of RAD51 complex inhibitor administered to a subject is at least 15%, at least 20%, at least 30%, at least 40%, or at least 50% less than the standard-of-care dose for the RAD51 complex inhibitor. In some embodiments, the dose of RAD51 complex inhibitor administered to a subject is 10%-50%, 10%-40%, 10%-30%, 10%-20%, 20%-50%, 20%-40%, 20%-30%, 30%-50%, 30%-40%, or 40%-50% less than the standard-of-care dose for the RAD51 complex inhibitor.

In some embodiments, the RAD51 complex inhibitor B02 is administered to a subject at a dose of 30 mg/kg, or at a dose of less than 30 mg/kg. In some embodiments, the RAD51 complex inhibitor B02 is administered to a subject at a dose of 45 mg/kg, or at a dose of less than 45 mg/kg. In some embodiments, the RAD51 complex inhibitor B02 is administered to a subject at a dose of 60 mg/kg, or at a dose of less than 60 mg/kg. In some embodiments, the RAD51 complex inhibitor B02 is administered to a subject at a dose of 10-100 mg/kg. In some embodiments, the RAD51 complex inhibitor B02 is administered to a subject at a dose of 10, 15, 20, 25, 30, 35, 40, 45, 50, 55, 60, 65, 70, 75, 80, 85, 90, 95, or 100 mg/kg.

In some embodiments, the RAD51 complex inhibitor RI-1 is administered to a subject at a dose of 30 mg/kg, or at a dose of less than 30 mg/kg. In some embodiments, the RAD51 complex inhibitor RI-1 is administered to a subject at a dose of 45 mg/kg, or at a dose of less than 45 mg/kg. In some embodiments, the RAD51 complex inhibitor RI-1 is administered to a subject at a dose of 60 mg/kg, or at a dose of less than 60 mg/kg. In some embodiments, the RAD51 complex inhibitor RI-1 is administered to a subject at a dose of 10-100 mg/kg. In some embodiments, the RAD51 complex inhibitor RI-1 is administered to a subject at a dose of 10, 15, 20, 25, 30, 35, 40, 45, 50, 55, 60, 65, 70, 75, 80, 85, 90, 95, or 100 mg/kg.

In some embodiments, the RAD51 complex inhibitor DIDS is administered to a subject at a dose of 30 mg/kg, or at a dose of less than 30 mg/kg. In some embodiments, the RAD51 complex inhibitor DIDS is administered to a subject at a dose of 45 mg/kg, or at a dose of less than 45 mg/kg. In some embodiments, the RAD51 complex inhibitor DIDS is administered to a subject at a dose of 60 mg/kg, or at a dose of less than 60 mg/kg. In some embodiments, the RAD51 complex inhibitor DIDS is administered to a subject at a dose of 10-100 mg/kg. In some embodiments, the RAD51 complex inhibitor DIDS is administered to a subject at a dose of 10, 15, 20, 25, 30, 35, 40, 45, 50, 55, 60, 65, 70, 75, 80, 85, 90, 95, or 100 mg/kg.

In some embodiments, a glycolytic inhibitor is administered to a subject at a dose that is lower than the standard-of-care dose of fludarabine. In some embodiments, a RAD51 complex inhibitor is administered to a subject at a dose that is lower than the standard-of-care dose of fludarabine. Fludarabine is a chemotherapy agent used in the treatment of leukemia and lymphoma, including chronic lymphocytic leukemia (CLL), non-Hodgkin's lymphoma, acute myeloid leukemia (AML), and acute lymphocytic leukemia (ALL). Fludarabine is a purine nucleotide analog which inhibits DNA synthesis by interfering with ribonucleotide reductase and DNA polymerases. Thus, cells which are rapidly proliferating, such as cancer cells, will be more affected by fludarabine treatment than cells which are not proliferating rapidly. The standard-of-care dosage for fludarabine in CLL is between 15-40 mg/m² and is administered by intravenous infusion. In some embodiments, the dose of glycolytic inhibitor and/or RAD51 complex inhibitor administered is between 15-40 mg/m². In some embodiments, the dose of glycolytic inhibitor and/or RAD51 complex inhibitor administered is less than 15-40 mg/m². In some embodiments, the dose of glycolytic inhibitor and/or RAD51 complex inhibitor administered is 5 mg/m², 10 mg/m², 15 mg/m², 20 mg/m², 25 mg/m², 30 mg/m², 35 mg/m², or 40 mg/m².

In some embodiments, the glycolytic inhibitor and the RAD51 complex inhibitor are administered in an amount effective to reduce the number of CD19⁺ cells in the subject relative to a control (e.g., baseline, prior to administration of the inhibitors, or following administration of only one of the inhibitors). CD19 is a surface protein used as a biomarker for normal and cancerous B cells. CD19⁺ B cells play an essential role in the adaptive immune response, and promote the recognition and clearance of antigens in the system and, in the process, creating immune cells for immune-surveillance. The number of CD19+ cells in a subject may be determined using any number of biomarker detection methods (e.g., using anti-CD19 antibodies).

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

The provided examples illustrate different components and methodology useful in practicing the present disclosure. The examples do not limit the claimed disclosure. Based on the present disclosure the skilled artisan can identify and employ other components and methodology useful for practicing the present disclosure.

### Example 1: 2DG Alleviated Tumor Burden in a Spontaneous Mouse Model of Lymphomagenesis

SJL/J mice spontaneously develop a hyperplastic disorder involving CD4+ T cell and B cells that resembles non-Hodgkin lymphoma and is evident after one year of age (27, 28). It is thought that activated CD4⁺ T-cells secreting interleukin 21 drive B-cells to transformation in this model (29). SJL/J mice deficient in CD8a and thus lacking CD8+ show significantly accelerated development of B-cell lymphomas, with no change in other aspects of their phenotype (30). Since the growth or maintenance of any tumor requires energy, and highly proliferative cells such as cancer cells depend on numerous modes of ATP production, including glycolysis, to meet their energetic demands (4), blocking glycolysis in cancer cells at the first steps following cellular glucose intake should, in theory, reduce tumor burden (6, 7).

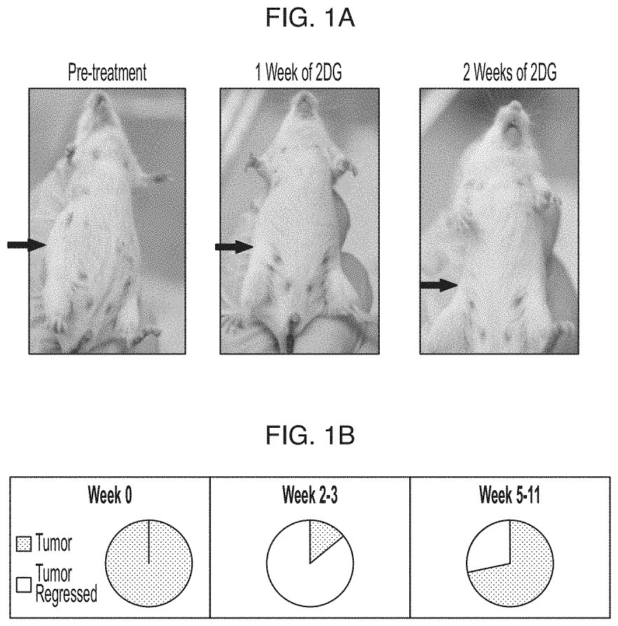

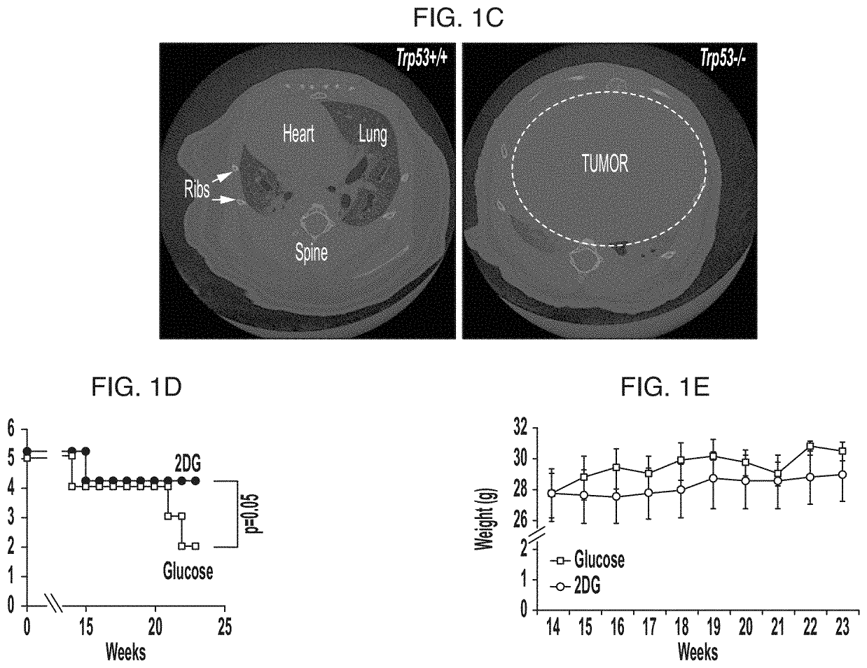

To test the extent to which inhibition of glycolysis by 2DG alleviated these spontaneously arising lymphomas, a cohort of SJL.CD8a−/− female mice were aged to 13 months of age and were monitored for signs of lymphoma development. Visible growth was most evident in cervical lymph nodes and in some cases in spleens (indicated by arrows, FIG. 1A). Once tumors were sufficiently large to palpate, mice were placed on water with 6 g/L of dissolved 2DG provided ad libitum and monitored weekly for tumor reduction. The average mouse consumed approximately 4 mL of water each day; hence, based on the average weight of the mice, each mouse received a dose of 2DG of approximately 900 mg/kg/day. Photographs of the tumors were taken weekly to document disease regression, and the time points, in weeks, at which the tumors visibly shrank or, later, reemerged, were recorded. Via gross observation of the mice, no adverse side effects of this treatment were observed, such as weight loss, lethargy, or lack of grooming, either shortly after treatment initiation, or at any time during the treatment.

Of the seven mice in this study, six showed evidence of tumor regression after two to three weeks of treatment (FIGS. 1A and 1B). However, in four of these six, the tumors returned within 5-11 weeks, despite continuation of the treatment. This significant regression, which was similar to what is observed in mouse models of solid cancer treated with 2DG, suggested that SJL lymphomas were partially responsive to relatively high therapeutic doses of a combination treatment for lymphoid cancers (reviewed in (10)).

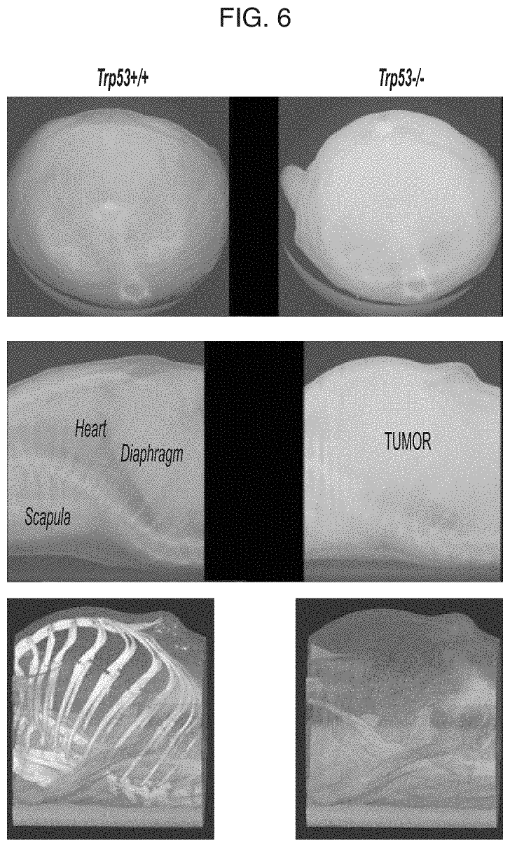

Next, a more homogeneous and acute spontaneously arising lymphoma was tested. In addition, the extent to which 2DG could affect a purely T-cell lymphoma was tested. To meet all of these criteria, a classic mouse model of T-cell cancer, the p53-deficient mouse (31) was used. The Trp53 gene codes for the p53 protein, and deficiency of this gene in mice lead to thymic lymphomas as early as 14 weeks of age (FIG. 1C and FIG. 6); because of this phenotype, the Trp53−/− mouse is considered a model of Li-Fraumeni Syndrome {Jacks, 1994 #134}. To test the effect of 2DG on these thymic lymphomas, C57BL6J. Trp53−/− mice were treated with either 2DG (200 μL of 2DG at 600 mM in DPBS (670 mg/kg)) or glucose, intraperitoneally (I.P.) three times weekly, starting at 14 weeks of age and continuing for 10 weeks.

Mice treated with 2DG were significantly protected from developing neoplasms compared to glucose-treated mice (FIG. 1D). Two notable adverse effects were observed with 2DG treatment delivered I.P.: first, upon injection, 2DG-treated mice showed inactivity for 10-60 minutes, and, second, as the experiment progressed, the 2DG-treated mice showed lower weight gain compared to glucose-treated mice, although the difference did not achieve significance (FIG. 1E).

Together, based on two different mouse models of spontaneous cancer, 2DG administered orally or I.P. alleviated both B- and T-cell tumor burdens. 2DG was also effective in shrinking tumors in SJL.CD8a−/− mice.

### Example 2: Lung Tumor PDX Models Indicated that Metabolic Differences and not Proliferation Determined Susceptibility to 2DG

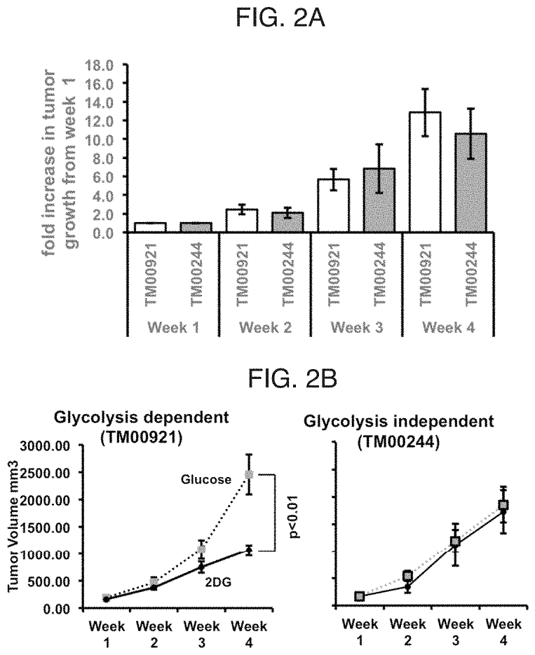

The effects of 2DG in human lung carcinoma PDX (patient-derived xenograft) models were eventuated. While it is understood that 2DG primarily affects glycolysis, studies have shown that 2DG can interfere with other systems, such as the cell cycle, independently of its effects on metabolism (8, 32, 33). Changes in the cell cycle would affect the proliferation of cancer cells, and would ultimately influence tumor growth. To test whether 2DG affects glycolysis independently of proliferation, two PDX human lung carcinomas (TM00244 and TM00921) with similar growth kinetics (FIG. 2A) but differing in the use of glycolytic pathways (Table 1) were tested.

TABLE 1
Pathway differences between TM00921
and TM00244 (INGENUITY/KEGG PATHWAY)

| | Number | |
|---|---|---|
| Pathway | of genes | p-Value |
| Metabolic pathways | 11 | 6.6E−2 |
| Arachidonic acid metabolism | 4 | 3.4E−3 |
| Drug metabolism-cytochrome P450 | 4 | 4.4E−3 |
| Metabolism of xenobiotics by cytochrome P450 | 4 | 5.6E−3 |
| Tyrosine metabolism | 3 | 1.3E−2 |
| Glutathione metabolism | 3 | 2.6E−2 |
| Chemical carcinogenesis | 3 | 5.9E−2 |
| Toll-like receptor signaling pathway | 3 | 9.5E−2 |
| Phenylalanine metabolism | 2 | 8.1E−2 |

A direct comparison of overall gene expression between the two carcinomas revealed that one carcinoma, TM00244, showed transcripts for alternative metabolic pathways as compared to the TM00921 carcinoma (Table 1). Tumors from these two carcinomas were xenografted into immunodeficient NOD.Scid.IL2R gamma null (NSG™) mice, and, after tumors of a measurable size (>50 mm³) were observed, mice of each model were treated with 200 μL of either 600 mM 2DG or glucose three times weekly via I.P. injection. After 4 weeks of treatment, TM00921-xenografted mice showed a significantly smaller tumor volume with 2DG treatment compared to glucose treatment, whereas TM00244-xenografted mice showed no difference in tumor size between 2DG- and glucose-treated mice (FIG. 2B), suggesting that inherent differences in metabolic pathway use—i.e., reliance on glycolysis or not—may impact the response to 2DG.

### Example 3: Glycolysis-Dependent B-Cell Cancers are Sensitive to 2DG

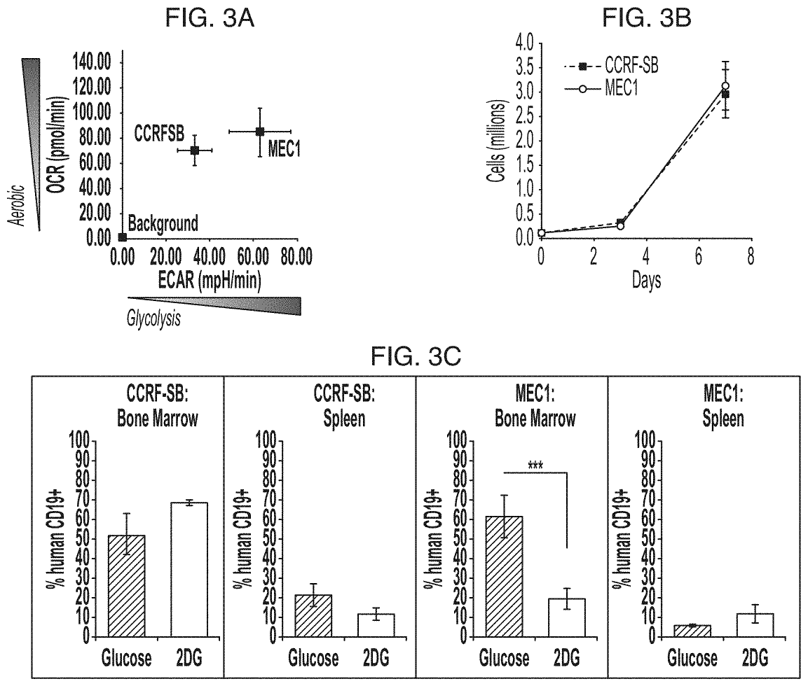

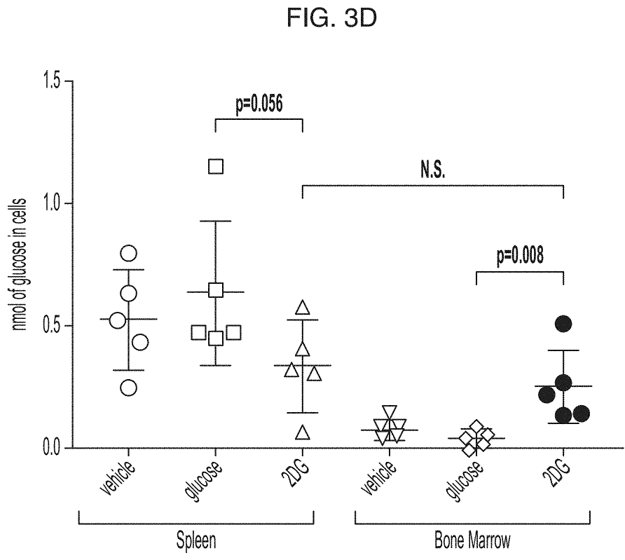

While the above experiments using PDX models gave a strong indication that lung tumors relying on glycolysis would be susceptible to 2DG therapy, the heterogeneity of PDX tumors can be a confounding variable, as each PDX tumor contains multiple cell types with different glycolytic demands (34, 35). To control for this cellular heterogeneity, and to extend the findings to human B-cell cancers, B-cell cancer cell lines were screened, and two lines, CCRF-SB and MEC1, were obtained that shared similar aerobic respiration rates but differ in their use glycolysis as a source of energy (FIG. 3A), and yet had similar proliferation rates ex vivo (FIG. 3B) (36, 37).

These data allowed the hypothesis that MEC1 cells, which were more glycolysis-dependent than are CCRF-SB cells, would be more sensitive to 2DG. To test that hypothesis, the two cell lines were tested for 2DG sensitivity in vivo: immunodeficient NOD.Cg-Rag1^{tm1Mom}Il2rg^{tm1Wjl}/SzJ (NRG™) mice were xenografted with either MEC1 or CCRF-SB cells, and the mice were then treated with 200 μL of 600 mM 2DG or glucose I.P. three times per week for two weeks. Both MEC1 and CCRF-SB cells migrate to the spleen and bone marrow, and since these mice express CD19+ B-cells, the tumor burden can be measured terminally in these organs by anti-human CD19 staining via flow cytometry after euthanasia at the end of the treatment period.

In contrast to CCRF-SB cells, which showed no significant response to 2DG in spleens or bone marrow, MEC1 cells in the bone marrow showed significant reductions in numbers with 2DG treatment (FIG. 3C). Interestingly, while MEC1 cells in the bone marrow showed sensitivity to 2DG, those in the spleen did not (FIG. 3C). This latter result may indicate that there might be site-specific differences in the response of MEC1 cells to 2DG, due to either exposure or accumulation of 2DG in the bone marrow. Overall, the results suggested that glycolysis-dependent B-cell cancers were more sensitive than glycolysis-independent B-cell cancers to 2DG, and that MEC1 cells in different sites may also differ in their sensitivity to 2D (B-lymphoid tumors that utilize glycolysis were susceptible to 2DG).

### Example 4: The RAD51 Inhibitor DIDS Reduced the Splenic Tumor Burden of a Chronic Lymphocytic Leukemia (CLL) Xenografted Cell Line

As noted above, AID generates immunoglobulin locus-independent DNA breaks throughout the genome that are repaired by XRCC2- and RAD51-dependent homologous recombination (15, 16).—It was previously shown that DIDS could specifically target AID-positive neoplastic cell lines in tissue culture (17). Importantly, DIDS also targeted AID-positive, but not AID-negative, human CLL cells from patients ex vivo (17). To test the extent to which DIDS could target a xenografted AID-positive CLL cell line in vivo, the glycolytic MEC1 B-cell line was utilized, which can be quantified by CD19 expression ex vivo (36).

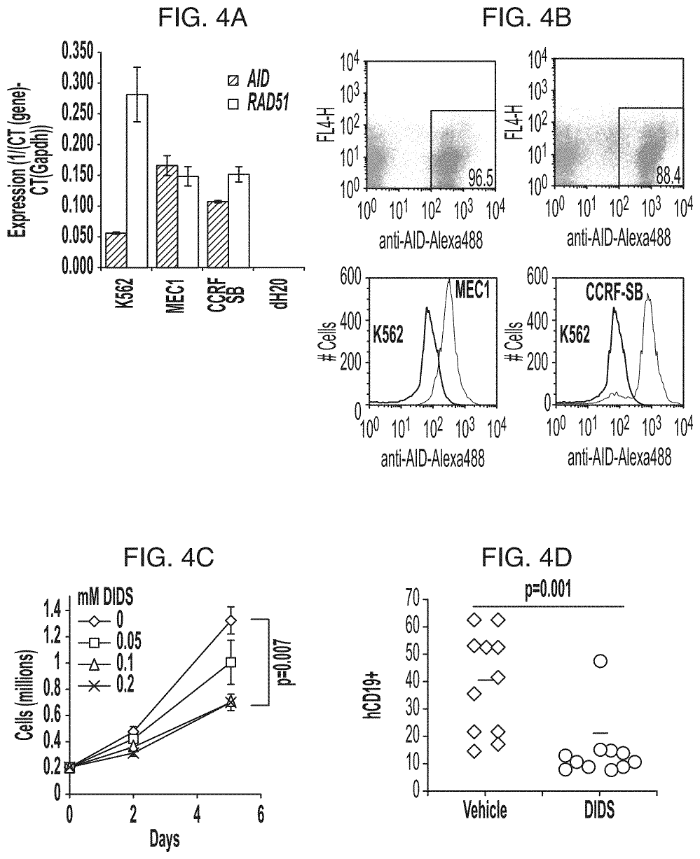

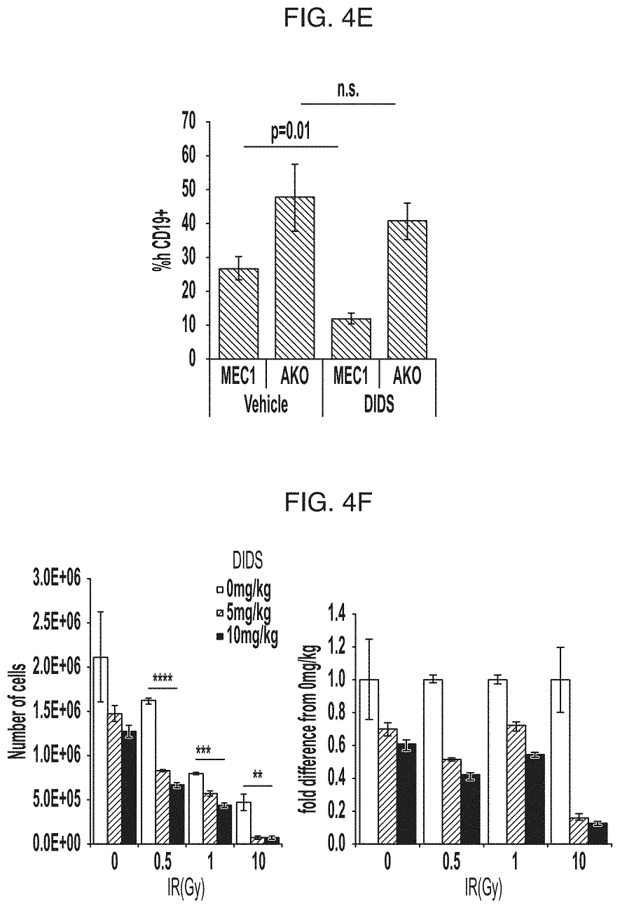

AID positivity of MEC1 B-cells was confirmed, using reverse transcriptase (RT)-PCR, and then compared AID expression of MEC1 B-cells with that of K562 cells, an AID-deficient cell line, and of CCRF-SB, an AID-positive ALL cell line. To control for the amount of RNA input, the samples were also tested for the control gene Gapdh. Results showed that MEC1 B-cells constitutively express AID (FIG. 4A). AID was also detected by intracellular staining of AID and flow cytometry (FIG. 4B). To test the sensitivity of MEC1 B-cells to DIDS ex vivo, DIDS was titrated from 0 to 0.2 mM, with 2×10⁵ MEC1 B-cells. Cytotoxic effects of DIDS on MEC1 B-cells were seen as early as 5 days at 0.05 mM, and a significant effect was observed with doses at 0.1 and 0.2 mM after 5 days (FIG. 4C).

Because DIDS was effective in reducing numbers of MEC1 B-cells ex vivo, its effectiveness was tested in vivo. 2×10′ MEC1 cells were xenografted into immunodeficient NRG™ mice for two weeks, and mice were then treated either with DIDS at 50 mg/kg or with a 0.1 M potassium bicarbonate/PBS vehicle, once per week for an additional two weeks. NRG™ mice were used because they were deficient in CD19 cells, allowing quantification of MEC1 B-cells by staining spleen cells for human CD19, as the spleen is a homing organ for MEC1 B-cells, all of which are positive for CD19. It was observed that, despite the wide range of xenograft capabilities among the tested mice, 10/11 mice treated with DIDS showed a significant (p=0.001) reduction in the number of MEC1 cells in the spleen (FIG. 4D).

### Example 5: The Therapeutic Effect of DIDS on MEC1 Cells Required AID

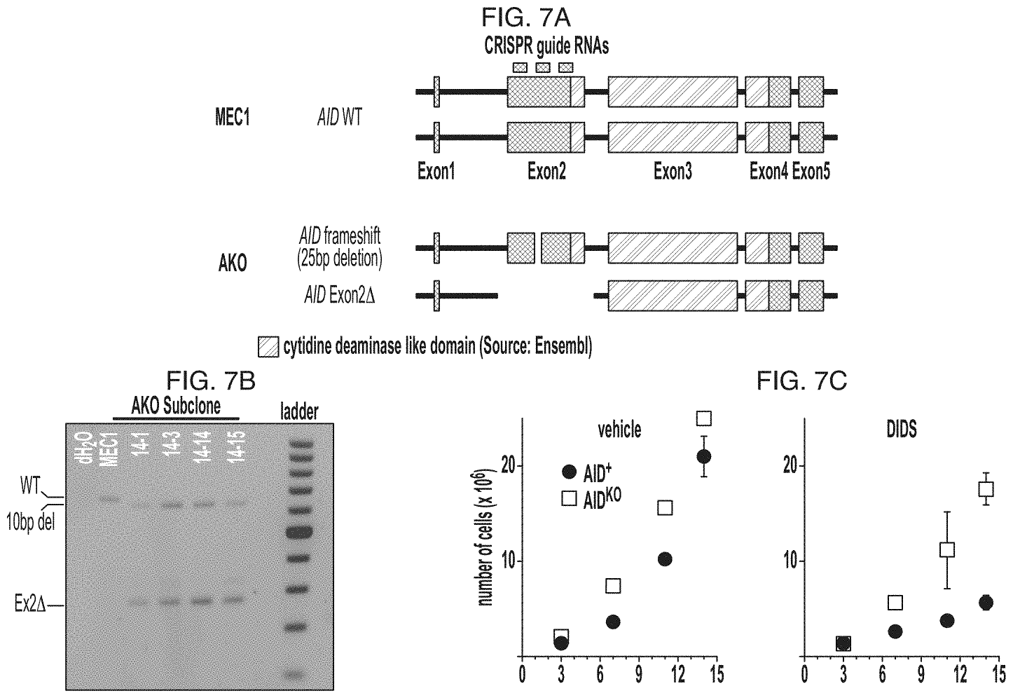

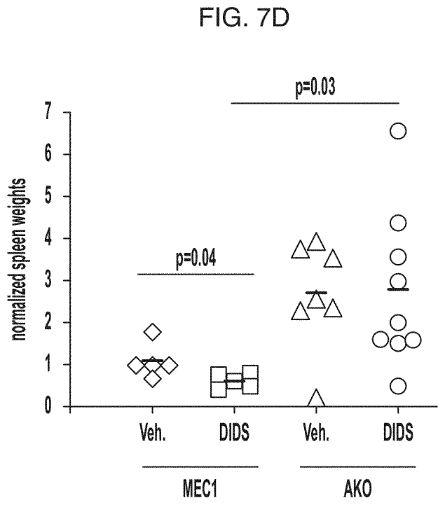

To directly determine if the effect of DIDS was dependent on AID, three CRISPR guides were generated that targeted exon 2 of AID (FIG. 7A). This exon was targeted because it is the first exon sufficiently large for the efficient design of CRISPR guides. The guides were transfected into MEC1 cells, and the cells were cloned by limiting dilution. One clone (Clone #14) that grew at a rate similar to the parental cell line, MEC1 was subcloned into four subclones (Clones #14-1, 14-3, 14-14 and 14-15). Upon sequencing, it was discovered that they all had a 25-base pair deletion in exon 2 of one allele and a complete deletion of exon 2 on the other (FIGS. 7A and 7B).

Theoretically, this would result in a truncated, non-functional AID, which was termed AID knock-out MEC1 (AKO) cells. To test the dependence of DIDS on AID, AKO cells were treated with DIDS and compared their growth with that of parental MEC1 cells. The impact of DIDS on the proliferation of AKO cells was significantly different than its impact on proliferation of parental MEC1 cells (FIG. 7C). Moreover, unlike the parental MEC1 cells, AKO cells grew at a similar rate regardless of whether the cells were treated with vehicle or DIDS (FIG. 7C). To test whether AID sufficiency was required for DIDS efficacy in vivo, AKO and parental cells were xenografted in NRG™ mice, and mice were treated with 50 mg/kg of DIDS for two weeks. Compared to parental xenografted MEC1 cells, AKO cells were more resistant to DIDS as measured by the percentages of human CD19 cells (FIG. 4E) and by spleen weights (FIG. 7D). Thus, the therapeutic effect of DIDS in MEC1 xenografts depended on AID.

The results above indicated that DIDS affects AID-initiated breaks in vivo. Whether DIDS could block repair of AID-independent double-strand breaks (frank or single-strand staggered double-strand breaks) in vivo was determined. A common way to generate double-strand breaks is via ionizing radiation (1, 38, 39). For this, AID-null mice were treated with different concentrations of DIDS (0, 5, or 10 mg/kg) for eight hours, and then harvested splenocytes and treated them with different doses of IR (0, 1, 5, 10 Gy). Eighteen hours after IR, the numbers of live white blood cells (CD45+ Ter119-PI−) were measured by flow cytometry.

Results showed that IR alone results in decreased survival of the cells; however, in the presence of DIDS, the effect is significantly greater (FIG. 4F). This effect was particularly evident at 10 Gy when the cell data are normalized to the 0 mg/Kg DIDS-treated data, where even a tenth of the dose of DIDS can synergistically enhance the severity of cell ablation (FIG. 4F). These data indicated, that while DIDS blocked repair of AID-initiated DSBs, it also blocked repair of IR-induced DSBs. Importantly, the results indicated that DIDS, or any RAD51 inhibitor, had the potential to be paired with ionizing radiation to enhance the therapeutic effect of the IR alone.

### Example 6: Rapid Synergistic Effect of Glycolytic Blockade and RAD51 Inhibition on MEC1 Cells Ex Vivo and In Vivo

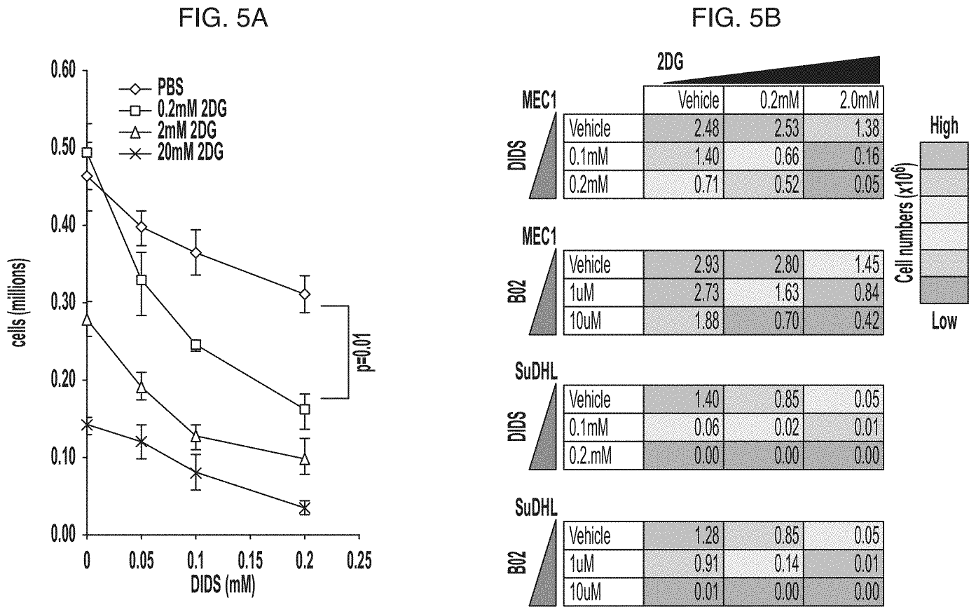

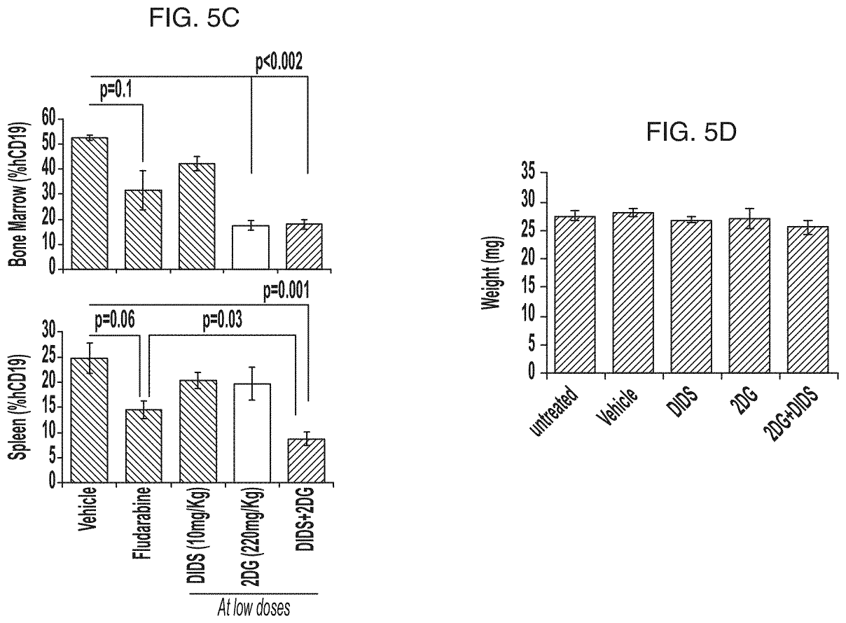

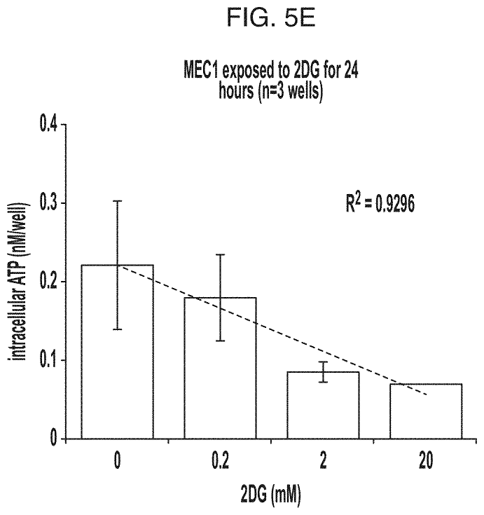

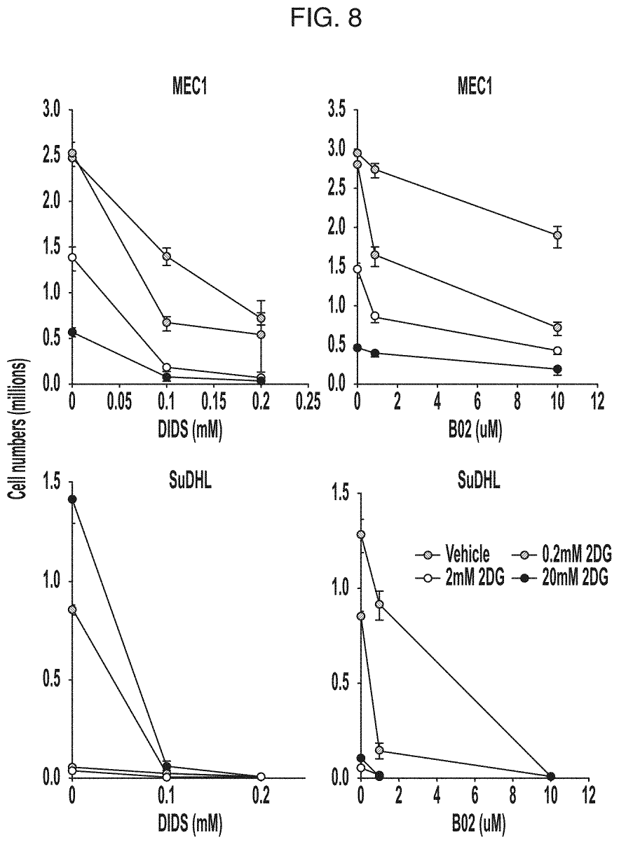

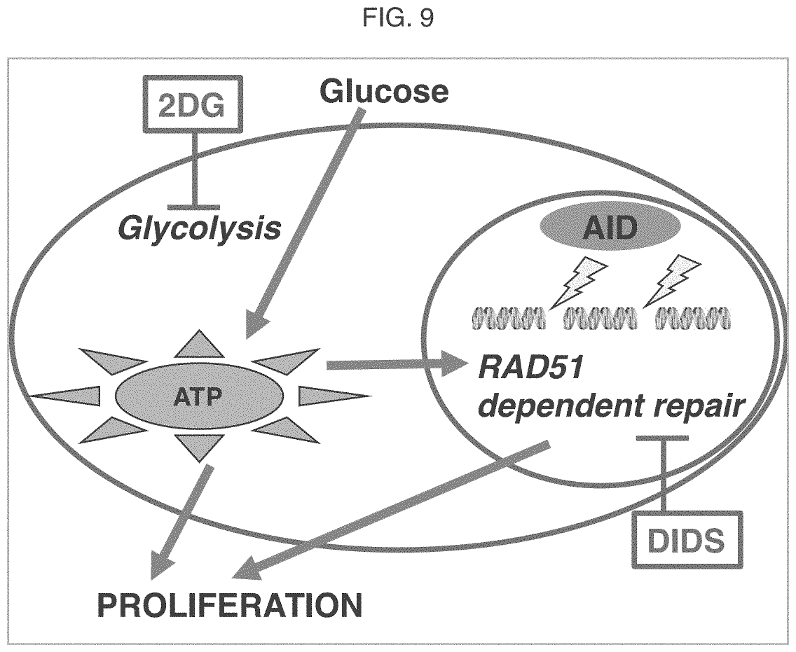

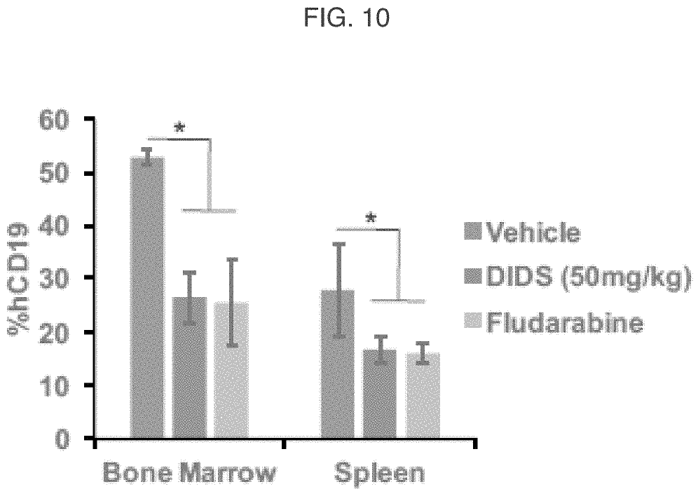

Having found that RAD51 inhibition by DIDS was improved by adjunct therapy such as radiation, it was tested whether the combined use of DIDS and 2DG would have a synergistic effect on AID-positive neoplasms, both in vitro and in vivo. It was reasoned that RAD51 function requires ATP and that, therefore, a reduction in the pool of ATP should decrease the efficiency of RAD51-dependent repair and enhance the cytotoxic effect of DIDS on rapidly proliferating AID+ neoplastic cells (39, 40) (FIG. 8A-8D). To test this possibility, 2DG was titrated with DIDS on MEC1 cells, since MEC1 cells are sensitive to both 2DG and DIDS. By day 2 there was a reduction of cell numbers as the concentration of DIDS was increased (FIG. 5A), consistent with the earlier results (FIG. 4C). However, the addition of 0.2 mM of 2DG, a concentration that does not have an effect on its own (y-axis, FIG. 5A), enhanced the cytotoxic effect of DIDS (FIG. 5A).

From the slopes of the titration, it was be surmised that 2DG had a synergistic effect with DIDS, not only with respect to the lower concentrations of DIDS required to elicit an effect, but also kinetically, since the effects were seen by 48 hours, in contrast to 5 days (FIG. 5A versus FIG. 4C). In order to determine the extent to which this 2DG-mediated synergistic effect held for any AID-positive cell treated with any RAD51 inhibitor, a different RAD51 inhibitor, B02 (Sigma) was used, on MEC1 and on another AID positive cell line, SuDHL cells. In addition, DIDS was tested on SuDHL cells. In each experiment, RAD51-inhibitor treatment (B02 or DIDS) with 2DG treatment was combined. Together, results of the two experiments and the earlier experiment treating MEC1 cells with DIDS and 2DG (FIG. 5A) showed that treatment of either of two AID-positive cell lines with 2DG had a similar synergistic effect on the efficacy of two different RAD51 inhibitors (FIG. 5B and FIG. 8). Importantly, the results also suggested that the combination of 2DG with any RAD51 inhibitor would have a synergistic therapeutic effect on any AID-positive cancer.

To test whether this synergistic effect at low doses can be recapitulated in vivo, MEC1-xenografted mice were treated with either DIDS alone, 2DG alone, or DIDS and 2DG in combination, all at low concentrations, or with a positive or a negative control. Mice were treated I.P. three times weekly for two weeks. 1) mice were dosed I.P. with 10 mg/kg DIDS, a fifth of the dose required to elicit an effect in vivo (FIG. 4D); 2) mice were treated I.P. with 220 mg/kg of 2DG or glucose; a third of the dose of 2DG required for an effect (FIG. 3C); 3) mice were treated with both DIDS and 2DG, at the same doses used in the first two groups; 4) as a positive control, mice were treated with fludarabine, a compound frequently used to treat CLL, at 35 mg/kg, a dose commonly used to elicit an effect; and 5) as a negative control, mice were treated with vehicle consisting of glucose in PBS (41). Following the 2-week treatment period, bone marrow and spleens were assessed for tumor burden, by determining the percentages of human CD19-positive cells via flow cytometry (FIG. 5C).

In the bone marrow (FIG. 5C), the combination DIDS/2DG treatment resulted in a significant (p<0.002) decrease in tumor burden compared to vehicle, and also a decrease compared to treatment with fludarabine, although this decrease was not statistically significant (p=0.26). Interestingly, in the bone marrow, 2DG alone elicited as much effect as the combination treatment (FIG. 5C). In the spleen, the combination treatment produced the best outcome compared to either compound separately or vehicle (p=0.001), and even performed significantly better than fludarabine (p=0.03) (FIG. 5C). Together, these data suggest that a combination RAD51-inhibitor/glycolysis-inhibitor treatment at lower doses was more effective in treating MEC1 cells in vivo than the current standard of treatment, fludarabine, indicating an effective means of treating cancers while minimizing the possibility of adverse side effects.

Furthermore, at these reduced doses of DIDS and 2DG, the common adverse effects of these treatments, including change in weight, were not observed, as at the end of the treatment regimen the weights of the treated mice were similar to those of the untreated or vehicle-treated mice (FIG. 5D). Together, these results indicated that 2DG in combination with the RAD51 inhibitor DIDS can be administered safely at reduced doses of each compound to produce a cytotoxic effect on the cancer that is more effective compared to treatment with DIDS alone, while eliciting no adverse effects, which is the best outcome for the treatment of any disease.

Also, in in vivo experiments by using 2DG and DIDS in combination, the dose of 2DG was reduced four-fold and the dose DIDS reduced five-fold, which reduced the tumor burden in chronic lymphocytic leukemia (CLL) cell line-xenografted mouse models more effectively than with fludarabine, a standard-of-care treatment.

Shown herein, 2DG monotherapy administered at high doses was efficacious in treatment of two different spontaneous mouse models of lymphoma: the SJL/J model of non-Hodgkin lymphoma and the Trp53−/− model of T cell lymphoma. Furthermore, high doses of 2DG showed greater efficacy in glycolysis-dependent human PDX lung cancers and B-cell line lymphoma xenografts than they did in those that were not strictly reliant on glycolysis. Together, these findings support the concept that glycolysis-dependent tumors can be targeted by 2DG. However, the dose of 2DG required to elicit an effect, combined with the transience of the tumor reduction/resistance (FIGS. 1A and 1B), suggested that 2DG alone is limited as an effective cancer therapy.

While, as stated above, the combination of 2DG with other therapies has shown the promise of synergistic effect, there is no cancer-cell specificity for any of the second compounds—most of them target basic functions such as DNA replication, cell cycle or transcription (10). Therefore, reducing the amount of 2DG, as is the norm in combination therapies, and combining it with a therapy that is targeted more specifically at cancer cells than at normal cells, could lead to the development of a more effective therapy, as specific targeting of cancer cells will minimize adverse effects.

In this study, the specific inhibition of RAD51, via DIDS, is combined with the generalized anti-glycolytic effect of 2DG, each at a lower dose than is used when the compound is administered alone, with the overall goal of narrowing the specificity of an anti-glycolytic inhibitor such that it impacts only cancer cells. This study has demonstrated that simultaneous inhibition of glycolysis and RAD51-dependent repair can augment the synthetic lethal effect specifically on AID+ cancers. The fact that about half of all CLLs and non-lymphoid tumors express AID(17, 18, 20, 21, 23) indicates that this therapy could be used for non-lymphoid cancers.

The observation that the combination of both glycolytic blockade and RAD51 inhibition lead to enhanced efficacy against tumorigenesis can be explained in two ways: First, ATP is required for multiple processes other than repair. Thus, reducing glycolysis by 2DG weakens the cancer cell, regardless of the action of DIDS. This was observed in the bone marrow in the MEC1 cell-xenografted study where there was no difference in the tumor burden with or without DIDS. Second, in the spleen, the presence of DIDS made a significant difference, and the two compounds showed a synergistic effect.

Lastly, simultaneous blocking of glycolysis was observed, with 2DG, and inhibition of RAD51, with DIDS, not only has a synergistic effect, but also resulted in a substantially more rapid effect compared to the use of DIDS alone. This advantage could be translated into a shorter treatment time for chemotherapy, which could lead to less severe adverse effects, lower costs, and a smaller chance that a tumor will develop resistance against the treatment.

In summary, combining glycolytic blockade using 2DG, and RAD51 inhibition using DIDS, at lower doses of each compared to the doses used when administered alone, synergistically reduced the numbers of AID-positive cancer cells both ex vivo and in vivo. The results presented herein suggested that this approach could be used with tumors that are not AID-positive, as RAD51 is used to repair fork collapses in rapidly replicating cells as well as to repair double-strand breaks (48, 49). Furthermore, while AID has been implicated as a tumor-promoting gene in a number of cancers, a modification of the combination therapy approach described herein could potentially be effective in homologous recombination-negative cancers such as BRCA mutants, in which AID is unlikely to play a tumorigenic role (50).

Specifically, patients could be administered recombinant AID and 2DG simultaneously. This could potentially result in a lethal combination in the cancer cell through administration of a clastogenic protein—AID—together with the creation of conditions for reduced levels of homologous recombination via depletion of the ATP pool. This combination could expand application of the therapy concept discussed herein to AID-negative cancers. Taken together, data provided herein demonstrate that the combination of AID, RAD51 inhibition, and glycolysis blockade was a beneficial combination for treating cancers while minimizing the potential for adverse effects, due to the cell specificity and kinetics, and the lower doses required to elicit a lethal effect on cancer cells.

### Materials and Methods

Mice

C57BL/6J (Cat. no. JR000664, JAX), C57BL/6J.129S2. Trp53−/− (Cat. no. 002101), NRG™ (Cat. no.007799, JAX), NSG′ (Cat. no. 005557, JAX), and SJL.Cd8a−/− (JR.004023, private strain: Derry Roopenian) mice used in this study were bred and housed at The Jackson Laboratory (Bar Harbor, Me.). Mice were provided with food and water ad libitum and were housed on a 14-hour light, 10-hour dark cycle. All procedures were approved by The Jackson Laboratory Animal Care and Use Committee (ACUC).

Female SJL.CD8a−/− mice were obtained from the Jackson Laboratory. Mice were aged and checked weekly for splenic or lymphatic tumors. Once significantly large tumors were observed, the mice were given water containing 6 g/L of 2-Deoxy-D-Glucose (2DG) ad libitum. Tumors were monitored and photographed weekly for signs of disease regression or progression.

### Cells

Human peripheral blood acute B-lymphoblastic leukemia cells (the CCRF-SB cell line (ATCC)); human chronic lymphocytic leukemia cells (the MEC1 cell line (Cat. no. ACC 497, DSMZ)); AID-positive peritoneal effusion B-lymphoblast SUDHL-4 cells; and K562 myeloid leukemia cells were cultured according to manufacturer's/donor's recommendations. Cell viability counts were done on the Countess II Automated Cell Counter (Invitrogen) according to manufacturer's recommendations.

### Generation of AID KO MEC1 Cell Line

AID knock out (AKO) MEC1 cell lines were developed by targeting Exon 2 of AID with the following guide RNAs: CTTGATGAACCGGAGGAAG (SEQ ID NO: 1), GTCCGCTGGGCTAAGGGTC (SEQ ID NO: 2), GTGCTACATCCTTTTCAC (SEQ ID NO: 3). These guides were cloned into the Cas9-EGFP vector, pX330 (Addgene, Cat. no. 66582). These vectors were nucleofected into MEC1 cells using Amaxa Cell Line Nucleofector Kit V using program X-001, and according to manufacturer's instructions (Lonza, Cat. no. VACA-1003). Nucleofected cells were sorted for GFP positivity and cloned by limited dilution to generate AKO cell lines. Two independent AKO cell lines (14-1 and 14-3) were confirmed for AID nullizygosity using both genomic and transcript PCRs.

Compounds

2-deoxy-D-glucose (Cat. no. D8375), the RAD51 inhibitor B02 (Cat. no. SML0364), and fludarabine phosphate (Cat. no. 1272204) were obtained from Sigma-Aldrich. DIDS was obtained from ChemCruz (Cat. no. sc-203919).

Structures are shown below:

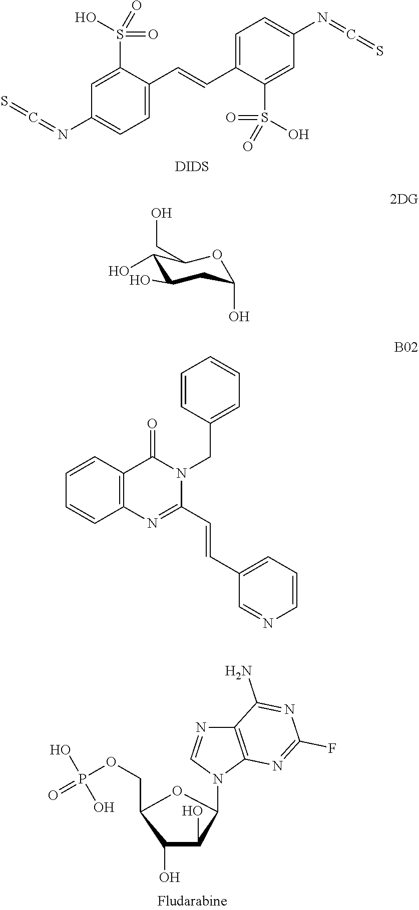

Quantification of AID and RAD51

Total RNA was extracted from tissue samples using the RNeasy Mini Kit according to manufacturer's protocol (QIAGEN, Cat. no. 74104).

PDX Xenografts and Treatments

Patient-derived xenograft (PDX) tumors were obtained from the JAX PDX Resource. Tumor fragments were minced separately and xenografted subcutaneously in an NSG mouse to establish P1. Upon growth of the tumor fragment, the tumor was harvested, minced, and xenografted in multiple mice to establish P2 mice. All experiments were conducted on P2 or higher mice that arose from engraftment from one of multiple tumor fragments.

Xenograft Studies

Human cancer cell lines were xenografted into NRG mice in the following way: Cells grown in flasks were given fresh media the night before xenograftment and seeded at 106 cells per mL. The next day, the cells were washed twice with DPBS without calcium or magnesium and resuspended to 1-2×10⁸ cells per mL of DPBS at room temperature and immediately injected via tail vein at 100 μL per mouse. The cells were given 7-10 days to fully xenograft, after which tumor load in the blood was assessed via flow cytometry (FACSCaliber II) for human CD19+ cells (Antibody cat no. 555413, BD Parmingen). Mice with similar tumor loads were assigned to different treatment groups to decrease bias and ensure more even tumor load across treatments. Mice were treated as described in the Results section for two weeks unless otherwise indicated. Upon euthanasia, the spleen and the bone marrow were harvested and stained for human CD19, and the cells were measured by flow cytometry on the FACSCaliber II. Analysis of the flow data was done by Flowjo version 8.8.7 (BD Pharmingen).

Xenografts were done as follows: NOD. Rag1−/− IL-2rg−/− (NRG) mice obtained from the Jackson Laboratory were xenografted for one to two weeks with Non Hogdkin's Leukemia CCRFSB, Chronic lymphocytic leukemia MEC1 cells or MEC1 cells carrying the luciferase gene (MEC1_LUC). Mice were then randomly assigned to treatment or vehicle cohort, and treated for two weeks.

Treatment of the Spontaneous Tumor Models with 2DG

SJL.CD8a−/− female mice were aged and monitored for signs of tumor development weekly after reaching 6 months of age. Tumors were found on mice in between 7 to 12 months of age. Once tumors had reached a clearly visible size, the mice were given water with 6 g/L of dissolved 2DG provided ad libitum. This concentration of 2DG in the water supplied the mice with a daily dose of approximately 960 mg/kg of 2DG. Mice were monitored weekly to determine changes in tumor mass. Photographs of the tumors were taken weekly to gauge whether the tumor was shrinking, expanding, or maintaining their size and to provide an estimate of the overall health of the mice. Treatments lasted for 11 weeks or until the tumors returned and mice became too sick to continue in the study. The treatment of one mouse was carried out to week 16 to determine what, if any, side effects would occur with prolonged 2DG exposure. This tumor never returned and no adverse effects were noted. Dates on which tumor shrinkage was observed, as well as dates on which tumor reemergence was observed, were recorded.

Statistics

All statistical parameters were calculated using EXCEL version 15.4. Error bars are standard error of the mean (SEM) and p-values are calculated by student t-test, unless indicated otherwise.

Student t-test was used to calculate p-values: *=p≤0.1 **=p≤0.05 ***=p≤0.01

Detection of AID and RAD51 by qPCR

1% β-mercaptoethanol in Buffer RLT was added to the tissue at a ratio of 100 mg/1.25 mL. Synthesis of cDNA was done using the RT² First Strand Kit following manufacturer's protocol (QIAGEN, Cat. no. 330404). 500 ng of RNA was used when available; otherwise, the next-largest amount of RNA for the set of samples was used. For qPCR, oligonucleotides to detect GAPDH transcripts were 5′-GAGTCAACGGATTTGGTCGT-3′ (forward) (SEQ ID NO: 4) and 5′-TTGATTTTGGAGGGATCTGC-3′ (reverse) (SEQ ID NO: 5). Oligonucleotides to detect AICDA transcripts were 5′-TTCTTTTCACTGGACTTTGG-3′ (forward) (SEQ ID NO: 6) and 5′-GACTGAGGTTGGGGTTCC-3′ (reverse) (SEQ ID NO: 7). Oligonucleotides to detect RAD51 transcripts were 5′-CAACCCATTTCACGGTTAGAGC-3′ (forward) (SEQ ID NO: 8) and 5′-TTCTTTGGCGCATAGGCAACA-3′ (reverse) (SEQ ID NO: 9). For each sample, three 25-4, reactions were run of varying cDNA concentrations (10, 5, and 1 μL). The reactions were run on an Applied Biosystem Model 7500 thermocycler using RT² SYBR Green ROX qPCR Mastermix (QIAGEN, Cat. no. 330520). PCR conditions were 50° C. for 2 min, 95° C. for 10 min, followed by 40 cycles of 95° C. for 15 s, 60° C. 1 min.

Detection of AID by Flow Cytometry

Cells were fixed in 3% Neutral Buffered Formalin (Cat. no. MER 44991 GL, Mercedes Chemicals), 2% sucrose (Cat. no. S8501 Sigma-Aldrich) in DPBS (Cat. no. 14190250, ThermoFisher) at a concentration of one million per mL for 10 minutes in suspension at room temperature. The cells were then washed with DPBS by centrifugation (400×g×5 mins) and permeabilized with 0.1% Triton-X-100 (Cat. no. X100, Sigma-Aldrich) in DPBS, and washed once with DPBS. The cells were then incubated with 1 mL of 10% FBS in DPBS for 1 hour, and stained overnight at 4° C. with anti-AID antibody (ab93596, Abcam) at 1:100 in 0.1 mL, 10% FBS in DPBS. The cells were washed twice with DPBS, and incubated with a 1:1000 dilution of Alexa 488 goat anti rabbit IgG (Cat. no. A27016, ThermoFisher) in 0.1 mL of 10% FBS in DPBS for 1 hour at room temperature in the dark. The cells were washed twice with DPBS, and data were acquired using a FACScaliber II and were analyzed by Flowjo Version 8.8.7.

Metabolic Measurements

Aerobic and glycolytic measurements on cell lines were done on the Agilent Seahorse XFe96 Analyzer using the Agilent XF cell energy phenotype testing test kit according to the manufacturer's instructions (Cat. no. 103325-100).

MicroCT Imaging and Image Reconstruction

Micro Computed Tomography (μCT) was performed using a high-speed in vivo μCT scanner (Quantum GX, PerkinElmer, Hopkinton, Mass., USA). The images were acquired using a High Resolution Scan mode with a 4-min scan time. The X-ray source was set to a current of 88 μA, voltage of 90 kVp, and a 36 mm FOV for a 50 μm voxel size. Animals were anesthetized with 2% isoflurane via nose cone while imaging. Administration of anesthesia helped to minimize motion artifacts during scanning. Animals were recovered in a clean box with pine shavings placed on a 37° C. heating pad until fully mobile and then returned to their home cages.

The μCT imaging was visualized via 3D Viewer, existing software within the Quantum GX system. The greyscale image slices were selected on the basis of internal landmarks such as ribs and spinal column so that images were generated in approximately the same location within each animal. These images were saved as JPEG files. Colored images were reconstructed using Image J32 (V1.49) or the PerkinElmer 3D Viewer application. Thresholds were used to visually determine optimal separation of the histogram into bone and soft tissue. In this way, the lungs and tumor could be viewed separately from the bone.

Gene Expression Analysis

Expression analyses of early JAX PDX oncology models were performed utilizing the ThermoFisher Scientific (formerly Affimetrix) GeneChip™ Human Genome U133 Plus 2.0 Array or the GeneChip™ Human Gene 1.0 ST Array. Arrays from all microarray-assayed models were processed with the AffyPLM R package, using quantile normalization and no background correction, and fitted to a simple model that treats the log intensity as a sum of array effect, probe effect, and residual. The array effect was the ‘summarized expression’ that was equivalent to the median polished value produced by standard RMA analysis. The differences between the normalized expression for each gene in models TM00244 and TM00921 were calculated to identify the genes that showed the greatest difference in expression between the models. The gene list was then exported into Ingenuity Pathway Analysis software, which revealed the effected pathways.

### REFERENCES

1. Berg J M, Tymoczko J L, Stryer L. Biochemistry, 5th Edition. New York: W H Freeman; 2002.
2. Foley G E L, H. Farber, S. Uzman, B. Adams, R. Studies on Human Leukemia Cells in Vitro. The Proliferation and Spread of Neoplastic Cells. Baltimore, Md.: The Williams & Wilkins Co; 1968. p. 65-97.
3. Gatenby R A, Gillies R J. Why do cancers have high aerobic glycolysis? Nat Rev Cancer. 2004; 4(11):891-9. Epub 2004/11/02. doi: 10.1038/nrc1478. PubMed PMID: 15516961.
4. Vander Heiden M G, Cantley L C, Thompson C B. Understanding the Warburg effect: the metabolic requirements of cell proliferation. Science. 2009; 324(5930):1029-33. Epub 2009/05/23. doi: 10.1126/science.1160809. PubMed PMID: 19460998; PMCID: PMC2849637.
5. Pelicano H, Martin D S, Xu R H, Huang P. Glycolysis inhibition for anticancer treatment. Oncogene. 2006; 25(34):4633-46. doi: 10.1038/sj.onc.1209597. PubMed PMID: 16892078.
6. Kim J W, Dang C V. Cancer's molecular sweet tooth and the Warburg effect. Cancer Res. 2006; 66(18):8927-30. Epub 2006/09/20. doi: 10.1158/0008-5472.CAN-06-1501. PubMed PMID: 16982728.
7. Chen Z, Lu W, Garcia-Prieto C, Huang P. The Warburg effect and its cancer therapeutic implications. J Bioenerg Biomembr. 2007; 39(3):267-74. Epub 2007/06/07. doi: 10.1007/s10863-007-9086-x. PubMed PMID: 17551814.
8. Muley P, Olinger A, Tummala H. 2-Deoxyglucose induces cell cycle arrest and apoptosisin colorectal cancer cells independent of its glycolysis inhibition. Nutr Cancer. 2015; 67(3):514-22. Epub 2015/03/10. doi: 10.1080/01635581.2015.1002626. PubMed PMID: 25751508.
9. Vijayaraghavan R, Kumar D, Dube S N, Singh R, Pandey K S, Bag B C, Kaushik M P, Sekhar K, Dwarakanath B S, Ravindranath T. Acute toxicity and cardio-respiratory effects of 2-deoxy-D-glucose: a promising radio sensitiser. Biomedical and environmental sciences: BES. 2006; 19(2):96-103. PubMed PMID: 16827179.
10. Zhang D, Li J, Wang F, Hu J, Wang S, Sun Y. 2-Deoxy-D-glucose targeting of glucose metabolism in cancer cells as a potential therapy. Cancer letters. 2014; 355(2):176-83. doi: 10.1016/j.canlet.2014.09.003. PubMed PMID: 25218591.
11. Stavnezer J, Schrader C E. IgH chain class switch recombination: mechanism and regulation. Journal of immunology. 2014; 193(11):5370-8. doi: 10.4049/jimmunol.1401849. PubMed PMID: 25411432; PMCID: 4447316.
12. Matthews A J, Zheng S, DiMenna L J, Chaudhuri J. Regulation of immunoglobulin class-switch recombination: choreography of noncoding transcription, targeted DNA deamination, and long-range DNA repair. Advances in immunology. 2014; 122:1-57. doi: 10.1016/B978-O-12-800267-4.00001-8. PubMed PMID: 24507154; PMCID: 4150736.
13. Teng G, Papavasiliou F N. Immunoglobulin somatic hypermutation. Annual review of genetics. 2007; 41:107-20. doi: 10.1146/annurev.genet.41.110306.130340. PubMed PMID: 17576170.
14. Robbiani D F, Bunting S, Feldhahn N, Bothmer A, Camps J, Deroubaix S, McBride K M, Klein I A, Stone G, Eisenreich T R, Ried T, Nussenzweig A, Nussenzweig M C. AID produces DNA double-strand breaks in non-Ig genes and mature B cell lymphomas with reciprocal chromosome translocations. Molecular cell. 2009; 36(4):631-41. doi: 10.1016/j.molcel.2009.11.007. PubMed PMID: 19941823; PMCID: 2805907.
15. Hasham M G, Donghia N M, Coffey E, Maynard J, Snow K J, Ames J, Wilpan R Y, He Y, King B L, Mills K D. Widespread genomic breaks generated by activation-induced cytidine deaminase are prevented by homologous recombination. Nature immunology. 2010; 11(9):820-6. doi: 10.1038/ni.1909. PubMed PMID: 20657597; PMCID: 2930818.
16. Hasham M G, Snow K J, Donghia N M, Branca J A, Lessard M D, Stavnezer J, Shopland L S, Mills K D. Activation-induced cytidine deaminase-initiated off-target DNA breaks are detected and resolved during S phase. Journal of immunology. 2012; 189(5):2374-82. doi: 10.4049/jimmunol.1200414. PubMed PMID: 22826323; PMCID: 3424338.
17. Lamont K R, Hasham M G, Donghia N M, Branca J, Chavaree M, Chase B, Breggia A, Hedlund J, Emery I, Cavallo F, Jasin M, Ruter J, Mills K D. Attenuating homologous recombination stimulates an AID-induced antileukemic effect. The Journal of experimental medicine. 2013; 210(5):1021-33. doi: 10.1084/jem.20121258. PubMed PMID: 23589568; PMCID: 3646491.
18. Gu X, Shivarov V, Strout M P. The role of activation-induced cytidine deaminase in lymphomagenesis. Curr Opin Hematol. 2012; 19(4):292-8. Epub 2012/04/21. doi: 10.1097/MOH.0b013e328353da3a. PubMed PMID: 22517589.
19. Matsumoto T, Shimizu T, Nishijima N, Ikeda A, Eso Y, Matsumoto Y, Chiba T, Marusawa H. Hepatic inflammation facilitates transcription-associated mutagenesis via AID activity and enhances liver tumorigenesis. Carcinogenesis. 2015; 36(8):904-13. Epub 2015/05/15. doi: 10.1093/carcin/bgv065. PubMed PMID: 25969143.
20. Nonaka T, Toda Y, Hiai H, Uemura M, Nakamura M, Yamamoto N, Asato R, Hattori Y, Bessho K, Minato N, Kinoshita K. Involvement of activation-induced cytidine deaminase in skin cancer development. J Clin Invest. 2016; 126(4):1367-82. Epub 2016/03/15. doi: 10.1172/JCI81522. PubMed PMID: 26974156; PMCID: PMC4811119.
21. Sapoznik S, Bahar-Shany K, Brand H, Pinto Y, Gabay O, Glick-Saar E, Dor C, Zadok O, Barshack I, Zundelevich A, Gal-Yam E N, Yung Y, Hourvitz A, Korach J, Beiner M, Jacob J, Levanon E Y, Barak M, Aviel-Ronen S, Levanon K. Activation-Induced Cytidine Deaminase Links Ovulation-Induced Inflammation and Serous Carcinogenesis. Neoplasia. 2016; 18(2):90-9. Epub 2016/03/05. doi: 10.1016/j.neo.2015.12.003. PubMed PMID: 26936395; PMCID: PMC5005261.
22. Shimizu T, Marusawa H, Endo Y, Chiba T. Inflammation-mediated genomic instability: roles of activation-induced cytidine deaminase in carcinogenesis. Cancer Sci. 2012; 103(7):1201-6. Epub 2012/04/04. doi: 10.1111/j.1349-7006.2012.02293.x. PubMed PMID: 22469133.
23. Sawai Y, Kodama Y, Shimizu T, Ota Y, Maruno T, Eso Y, Kurita A, Shiokawa M, Tsuji Y, Uza N, Matsumoto Y, Masui T, Uemoto S, Marusawa H, Chiba T. Activation-Induced Cytidine Deaminase Contributes to Pancreatic Tumorigenesis by Inducing Tumor-Related Gene Mutations. Cancer Res. 2015; 75(16):3292-301. Epub 2015/06/27. doi: 10.1158/0008-5472.CAN-14-3028. PubMed PMID: 26113087.
24. Casellas R, Basu U, Yewdell W T, Chaudhuri J, Robbiani D F, Di Noia J M. Mutations, kataegis and translocations in B cells: understanding AID promiscuous activity. Nat Rev Immunol. 2016; 16(3):164-76. Epub 2016/02/24. doi: 10.1038/nri.2016.2. PubMed PMID: 26898111; PMCID: PMC4871114.
25. Liu M, Duke J L, Richter D J, Vinuesa C G, Goodnow C C, Kleinstein S H, Schatz D G. Two levels of protection for the B cell genome during somatic hypermutation. Nature. 2008; 451(7180):841-5. doi: 10.1038/nature06547. PubMed PMID: 18273020.
26. Ratiu J J, Racine J J, Hasham M G, Wang Q, Branca J A, Chapman H D, Zhu J, Donghia N, Philip V, Schott W H, Wasserfall C, Atkinson M A, Mills K D, Leeth C M, Serreze D V. Genetic and Small Molecule Disruption of the AID/RAD51 Axis Similarly Protects Nonobese Diabetic Mice from Type 1 Diabetes through Expansion of Regulatory B Lymphocytes. Journal of immunology. 2017; 198(11):4255-67. doi: 10.4049/jimmunol.1700024. PubMed PMID: 28461573; PMCID: 5474749.
27. Lacoste-Collin L, Jozan S, Cances-Lauwers V, Pipy B, Gasset G, Caratero C, Courtade-Saidi M. Effect of continuous irradiation with a very low dose of gamma rays on life span and the immune system in SJL mice prone to B-cell lymphoma. Radiat Res. 2007; 168(6):725-32. Epub 2007/12/20. doi: 10.1667/RR1007.1. PubMed PMID: 18088184.
28. Thrush G R, Butch A W, Lerman S P. CD8 suppressor cell activity and its effect on CD4 helper cell-dependent growth of SJL/J B-cell lymphomas. Cell Immunol. 1989; 122(2):555-62. Epub 1989/09/01. PubMed PMID: 2569938.
29. Jain S, Chen J, Nicolae A, Wang H, Shin D M, Adkins E B, Sproule T J, Leeth C M, Sakai T, Kovalchuk A L, Raffeld M, Ward J M, Rehg J E, Waldmann T A, Jaffe E S, Roopenian D C, Morse H C, 3rd. IL-21-driven neoplasms in SJL mice mimic some key features of human angioimmunoblastic T-cell lymphoma. Am J Pathol. 2015; 185(11):3102-14. Epub 2015/09/13. doi: 10.1016/kajpath.2015.07.021. PubMed PMID: 26363366; PMCID: PMC4630166.
30. McPhee C G, Sproule T J, Shin D M, Bubier J A, Schott W H, Steinbuck M P, Avenesyan L, Morse H C, 3rd, Roopenian D C. MEW class I family proteins retard systemic lupus erythematosus autoimmunity and B cell lymphomagenesis. Journal of immunology. 2011; 187(9):4695-704. Epub 2011/10/04. doi: 10.4049/jimmunol.1101776. PubMed PMID: 21964024; PMCID: PMC3381364.
31. Jacks T, Remington L, Williams B O, Schmitt E M, Halachmi S, Bronson R T, Weinberg R A. Tumor spectrum analysis in p53-mutant mice. Curr Biol. 1994; 4(1):1-7. Epub 1994/01/01. PubMed PMID: 7922305.
32. Ralser M, Wamelink M M, Struys E A, Joppich C, Krobitsch S, Jakobs C, Lehrach H. A catabolic block does not sufficiently explain how 2-deoxy-D-glucose inhibits cell growth. Proceedings of the National Academy of Sciences of the United States of America. 2008; 105(46):17807-11. Epub 2008/11/14. doi: 10.1073/pnas.0803090105. PubMed PMID: 19004802; PMCID: PMC2584745.
33. Zhong D, Xiong L, Liu T, Liu X, Liu X, Chen J, Sun S Y, Khuri F R, Zong Y, Zhou Q, Zhou W. The glycolytic inhibitor 2-deoxyglucose activates multiple prosurvival pathways through IGF1R. The Journal of biological chemistry. 2009; 284(35):23225-33. Epub 2009/07/04. doi: 10.1074/jbc.M109.005280. PubMed PMID: 19574224; PMCID: PMC2749096.
34. Siolas D, Hannon G J. Patient-derived tumor xenografts: transforming clinical samples into mouse models. Cancer Res. 2013; 73(17):5315-9. Epub 2013/06/05. doi: 10.1158/0008-5472.CAN-13-1069. PubMed PMID: 23733750; PMCID: PMC3766500.
35. Okegawa T, Morimoto M, Nishizawa S, Kitazawa S, Honda K, Araki H, Tamura T, Ando A, Satomi Y, Nutahara K, Hara T. Intratumor Heterogeneity in Primary Kidney Cancer Revealed by Metabolic Profiling of Multiple Spatially Separated Samples within Tumors. EBioMedicine. 2017; 19:31-8. Epub 2017/04/15. doi: 10.1016/j.ebiom.2017.04.009. PubMed PMID: 28408240; PMCID: PMC5440602.
36. Stacchini A, Aragno M, Vallario A, Alfarano A, Circosta P, Gottardi D, Faldella A, Rege-Cambrin G, Thunberg U, Nilsson K, Caligaris-Cappio F. MEC1 and MEC2: two new cell lines derived from B-chronic lymphocytic leukaemia in prolymphocytoid transformation. Leukemia research. 1999; 23(2):127-36. PubMed PMID: 10071128.
37. Kaplan J, Mastrangelo R, Peterson W D, Jr. Childhood lymphoblastic lymphoma, a cancer of thymus-derived lymphocytes. Cancer Res. 1974; 34(3):521-5. Epub 1974/03/01. PubMed PMID: 4590920.
38. Haber J. Genome Instability: DNA repair and Recombination. 1st ed. New York: Garland Science; 2014 Dec. 16, 2013. 399 p.
39. Sung P. Catalysis of ATP-dependent homologous DNA pairing and strand exchange by yeast RAD51 protein. Science. 1994; 265(5176):1241-3. Epub 1994/08/26. PubMed PMID: 8066464.
40. Chi P, Van Komen S, Sehorn M G, Sigurdsson S, Sung P. Roles of ATP binding and ATP hydrolysis in human Rad51 recombinase function. DNA repair. 2006; 5(3):381-91. doi: 10.1016/j.dnarep.2005.11.005. PubMed PMID: 16388992.
41. Skarbnik A P, Faderl S. The role of combined fludarabine, cyclophosphamide and rituximab chemoimmunotherapy in chronic lymphocytic leukemia: current evidence and controversies. Ther Adv Hematol. 2017; 8(3):99-105. Epub 2017/03/02. doi: 10.1177/2040620716681749. PubMed PMID: 28246553; PMCID: PMC5305006.
42. Rebucci M, Michiels C. Molecular aspects of cancer cell resistance to chemotherapy. Biochemical pharmacology. 2013; 85(9):1219-26. doi: 10.1016/j.bcp.2013.02.017. PubMed PMID: 23435357.
43. Xu R H, Pelicano H, Zhou Y, Carew J S, Feng L, Bhalla K N, Keating M J, Huang P. Inhibition of glycolysis in cancer cells: a novel strategy to overcome drug resistance associated with mitochondrial respiratory defect and hypoxia. Cancer Res. 2005; 65(2):613-21. Epub 2005/02/08. PubMed PMID: 15695406.
44. Harper J W, Elledge S J. The DNA damage response: ten years after. Molecular cell. 2007; 28(5):739-45. doi: 10.1016/j.molcel.2007.11.015. PubMed PMID: 18082599.
45. Kawabata M, Kawabata T, Nishibori M. Role of recA/RAD51 family proteins in mammals. Acta medica Okayama. 2005; 59(1):1-9. PubMed PMID: 15902993.
46. Bibby M C. Orthotopic models of cancer for preclinical drug evaluation: advantages and disadvantages. Eur J Cancer. 2004; 40(6):852-7. Epub 2004/05/04. doi: 10.1016/j.ejca.2003.11.021. PubMed PMID: 15120041.
47. Pirmohamed M, Park B K. Genetic susceptibility to adverse drug reactions. Trends Pharmacol Sci. 2001; 22(6):298-305. Epub 2001/06/08. PubMed PMID: 11395158.
48. Atkinson J, McGlynn P. Replication fork reversal and the maintenance of genome stability. Nucleic acids research. 2009; 37(11):3475-92. Epub 2009/05/02. doi: 10.1093/nar/gkp244. PubMed PMID: 19406929; PMCID: PMC2699526.
49. Caddle L B, Hasham M G, Schott W H, Shirley B J, Mills K D. Homologous recombination is necessary for normal lymphocyte development. Molecular and cellular biology. 2008; 28(7):2295-303. doi: 10.1128/MCB.02139-07. PubMed PMID: 18212067; PMCID: 2268416.
50. Scully R, Chen J, Plug A, Xiao Y, Weaver D, Feunteun J, Ashley T, Livingston D M. Association of BRCA1 with Rad51 in mitotic and meiotic cells. Cell. 1997; 88(2):265-75. Epub 1997/01/24. PubMed PMID: 9008167.

What is claimed is:

1. A method comprising contacting a cell with a glycolytic inhibitor and a RAD51 complex inhibitor.

2. The method of claim 1, wherein the glycolytic inhibitor is 2-deoxy-D-glucose (2DG).

3. The method of claim 1 or 2, wherein the RAD51 complex inhibitor is selected from 4,4′-diisothiocyano-2,2′-stilbenedisulfonic acid (DIDS), (E)-3-benzyl-2-(2-(pyridine-3-yl) vinyl) quinazolin-4(3H)-one (B02), and 3-chloro-1-(3,4-dichlorophenyl)-4-(4-morpholinyl)-1H-pyrrole-2,5-dione (RI-1).

4. The method of any one of claims 1-3, wherein the cell is a cancer cell.

5. The method of claim 4, wherein the cancer cell is a glycolysis-dependent cancer cell.

6. The method of claim 5, wherein the cancer cell expresses activation induced cytidine deaminase (AID).

7. The method of any one of claims 4-6, wherein the cancer cell is a B-cell cancer cell.

8. The method of claim 7, wherein the B-cell cancer cell is a leukemia cell.

9. The method of claim 8, wherein the leukemia cell is a chronic lymphocytic leukemia (CLL) cell.

10. A composition comprising a glycolytic inhibitor and a RAD51 complex inhibitor.

11. The composition of claim 10, wherein the glycolytic inhibitor is 2-deoxy-D-Glucose (2DG).

12. The composition of claim 10 or 11, wherein the RAD51 complex inhibitor is selected from 4,4′-Diisothiocyano-2,2′-stilbenedisulfonic acid (DIDS), B02, and RI-1.

13. The composition of any one of claims 10-12, wherein each of the glycolytic inhibitor and the RAD51 complex inhibitor is formulated in the composition at a dose that is lower than a control standard-of-care dose, optionally wherein the control standard-of-care dose is a standard-of-care dose of fludarabine.

14. A method comprising administering to a subject a glycolytic inhibitor and a RAD51 complex inhibitor.

15. The method of claim 14, wherein the glycolytic inhibitor is 2-deoxy-D-Glucose (2DG).

16. The method of claim 14 or 15, wherein the RAD51 complex inhibitor is selected from 4,4′-Diisothiocyano-2,2′-stilbenedisulfonic acid (DIDS), B02, and RI-1.

17. The method of any one of claims 14-16, wherein the subject has a cancer.

18. The method of claim 17, wherein the cancer is a glycolysis-dependent cancer.

19. The method of claim 18, wherein the cancer expresses activation induced cytidine deaminase (AID).

20. The method of any one of claims 17-19, wherein the cancer is a B-cell cancer.

21. The method of claim 20, wherein the B-cell cancer is a leukemia.

22. The method of claim 21, wherein the leukemia is chronic lymphocytic leukemia (CLL).

23. The method of any one of claims 14-22, wherein the subject is a human subject.

24. The method of any one of claims 14-23, wherein the glycolytic inhibitor and the RAD51 complex inhibitor are administered sequentially.

25. The method of claim 24, wherein the glycolytic inhibitor and the RAD51 complex inhibitor are administered concurrently.

26. The method of any one of claims 14-25, wherein the glycolytic inhibitor and the RAD51 complex inhibitor are administered in an amount effective to reduce CD19+ cells in the subject relative to a control.

27. The method of any one of claims 14-26, wherein the glycolytic inhibitor and the RAD51 complex inhibitor are administered at doses that are lower than a control standard-of-care dose, optionally wherein the control standard-of-care dose is a standard-of-care dose of fludarabine.

28. A kit comprising a glycolytic inhibitor, a RAD51 complex inhibitor, and optionally one or more delivery device.
